(19) United States
(12) Patent Application Publication
(10) Pub. No.: US 2025/0017829 A1
(43) Pub. Date: Jan. 16, 2025

(54) A SUNSCREEN COMPOSITION

(71) Applicant: VeganicSKN Limited, Milton (AU)

(72) Inventor: Lev Mizikovsky, Rocklea (AU)

(21) Appl. No.: 18/710,089

(22) PCT Filed: Nov. 16, 2022
(86) PCT No.: PCT/AU2022/095001

§ 371 Date: May 14, 2024

(30) Foreign Application Priority Data
Nov. 16, 2021 (AU) 2021903680
May 13, 2022 (AU) 2022901287

(51) Int. Cl. A61K 8/26; A61K 8/27; A61K 8/97; A61Q 17/04

(52) U.S. Cl. CPC A61K 8/26; A61K 8/27; A61K 8/97; A61Q 17/04; A61K 2800/5922; A61K 2800/651; A61K 2800/805

(57) ABSTRACT

A sunscreen composition Disclosed herein are sunscreen compositions, including sunscreen base compositions, that comprise zinc oxide and aluminium oxide containing materials, in particular containing a calcinated mixture comprising aluminium oxide and zinc oxide, and methods for making the same.

### CROSS REFERENCE

The present application claims priority to Australian patent application numbers 2021903680, filed 16 Nov. 2021, and 2022901287, filed 13 May 2022, the entire contents of each of which are incorporated herein by reference in their entireties.

### FIELD OF THE INVENTION

The invention relates to sunscreen compositions, including sunscreen base compositions, that comprise zinc oxide and aluminium oxide containing materials, and methods for making the same. In particular, the invention relates to sunscreen compositions that may have improved UV-absorbing, UV-visible light scattering, and/or UV-visible light reflecting properties and/or that may exhibit increased SPF rating over time. However, it will be appreciated that the invention is not limited to this particular field of use.

### BACKGROUND

The following discussion of the prior art is provided to place the invention in an appropriate technical context and enable the advantages of it to be more fully understood. It should be appreciated, however, that any discussion of the prior art throughout the specification should not be considered as an express or implied admission that such prior art is widely known or forms part of the common general knowledge in the field.

As awareness increases of the risks associated with overexposure to the sun causing skin damage and skin cancer, sunscreens have become very widely used. Sunscreens are applied to the skin and desirably reflect and/or block UVA and UVB rays to prevent sunburn and skin damage. Many sunscreens include components such as organic chemical compounds that absorb UV light (such as octylmethoxycinnamate and/or octocrylene and avobenzone), inorganic compounds that absorb or reflect UV light (such as zinc oxide or titanium dioxide) and creams or carriers that enable the sunscreen to be easily and comfortably applied to the skin.

The effectiveness of sunscreens is typically measured using an SPF (sun protection factor) rating. The SPF rating is a measure of the fraction of sunburn producing UV rays that reach the skin. For example, “SPF 15” means that 1/15th of the burning radiation will reach the skin, assuming that the sunscreen is applied evenly at a dosage of 2 mg/cm2. A user can determine the effectiveness of the sunscreen by multiplying the SPF factor by the length of time it takes for him or her to suffer sunburn without sunscreen. Therefore, if a person develops sunburn in 10 minutes without wearing a sunscreen, the same person exposed to the same intensity of sunlight will avoid sunburn for 150 minutes if wearing sunscreen with an SPF of 15. However, SPF rating is considered to be an imperfect measurement of the degree of protection provided by a sunscreen. Consequently, most countries prescribe a maximum SPF rating that can be attributed to any sunscreen. For example, in Australia, the maximum SPF rating that can be attributed to any sunscreen is SPF 50+.

Many sunscreen formulations incorporate zinc oxide, which itself has a number of useful properties, including its UV absorbing, anticorrosion, and antimicrobial properties, that make it suitable for use in a variety of applications, including in sunscreen formulations.

For zinc oxide-based sunscreen formulations, the UV absorbance and associated sun-protection factor (SPF) rating generally increases with increasing concentration of zinc oxide. However, it can be challenging to formulate zinc oxide at high concentrations in stable formulations that retain their transparent properties when applied to the skin. Further, some regulatory bodies limit zinc oxide concentration in topical formulations, e.g. up to 25 wt. %, Accordingly, there is a limit to the amount of zinc oxide that can be incorporated into sunscreen formulations, which prevents higher SPF ratings from being achieved with zinc oxide-based sunscreen.

The SPF rating of a zinc oxide based sunscreen may be increased by adding alternative UV-absorbing ingredients. These are typically organic based UV-absorbers which can themselves cause dermatological issues and other toxicity issues. In contrast inorganic UV absorbers, such as zinc oxide, are classed by the US Food and Drug Administration (FDA) as “GRASE”, or “generally recognized as safe and effective”. There is a need to develop zinc oxide-based materials having improved properties, for example, providing improved UV protection, so that lower concentration zinc oxide-based formulations are able to produce higher SPF rated sunscreen without the need for additional organic based UV-absorbers. There is also a need to develop new zinc oxide-based materials with other advantageous properties that could overcome limitations for other application areas (e.g. improved properties of a sunscreen product, such as improved skin feel (e.g. soft touch focus), optimum transparency on skin, improved glow, dryness and antiperspirant properties (e.g. for sports sunscreens); or improved composite materials, such as glass composite materials for smart phone device screens having improved crack-resistant properties).

It is an object of the present invention to overcome or ameliorate one or more the disadvantages of the prior art, or at least to provide a useful alternative.

### SUMMARY OF THE INVENTION

The inventors of the present application have surprisingly discovered that by calcination of a mixture of aluminium oxide and zinc carbonate, they are able to produce a calcinated mixture comprising aluminium oxide and zinc oxide having improved UV-absorbing, UV-visible light scattering, and/or UV-visible light reflecting properties over a physical blend of the components.

Further, the inventors of the present application have surprisingly discovered that the addition of tropolone, or a derivate thereof, such as hinokitiol (e.g. from Thuja Plicata) can provide a significant increase in the initial SPF of a sunscreen composition when compared to an identical composition lacking the tropolone or derivative thereof (e.g. hinokitiol).

In a first aspect of the invention there is provided a sunscreen base, comprising:
a calcinated mixture comprising aluminium oxide and zinc oxide;
a dispersing and/or wetting agent; and
a carrier oil.

The following options may be used in conjunction with the first aspect, either individually or in any combination.

In certain embodiments, the sunscreen base further comprises tropolone, or a derivative thereof, such as hinokitiol. In certain embodiments, the tropolone derivative is selected from the group consisting of tropolone substituted with one or more groups selected from the group consisting of optionally substituted C₁₋₁₀ alkyl, optionally substituted C₂₋₁₀ alkenyl, optionally substituted C₂₋₁₀ alkynyl, optionally substituted cycloalkyl, optionally substituted cycloalkenyl, optionally substituted cycloalkynyl, optionally substituted heterocyclyl, optionally substituted C₁₋₁₀ heteroalkyl, optionally substituted C₂₋₁₀ heteroalkenyl, optionally substituted C₂₋₁₀ heteroalkynyl, amino, amido, optionally substituted C₁-C₁₀ ester, optionally substituted C₁-C₁₀ ketone, optionally substituted C₁-C₁₀ alkylamino, optionally substituted aryl, optionally substituted heteroaryl, ═O, ═S, cyano, CF₃, and halogen. In one embodiment, the tropolone derivative is tropolone substituted with one or more groups selected from the group consisting of C₁₋₁₀ alkyl; especially tropolone substituted with C₁₋₁₀ alkyl; more especially tropolone substituted with C₁₋₆ alkyl; most especially C₃ alkyl.

In certain embodiments, the sunscreen base comprises from about 0.05 wt. % to about 15 wt. % tropolone, or a derivative thereof, or from about 0.05 wt. % to about 10 wt. %, about 0.05 wt. % to about 5 wt. %, about 0.1 wt. % to about 3 wt. %, about 0.1 wt. % to about 2 wt. %, about 0.1 wt. % to about 15 wt. %, about 0.1 wt. % to about 10 wt. %, about 0.1 wt. % to about 1 wt. %, about 0.1 wt. % to about 0.5 wt. %, or about 0.1 wt. % to about 0.3 wt. % tropolone, or a derivative thereof. It may comprise, for example, about 0.05, 0.1, 0.2, 0.3, 0.4, 0.5, 1, 2, 3, 4, 4.5, 5, 5.5, 6, 7, 7.5, 8, 9, 10, 11, 12, or 15 wt. % tropolone, or a derivative thereof. In certain embodiments, the sunscreen base comprises from about 0.05 wt. % to about 5 wt. %, optionally from about 0.1 wt. % to about 1 wt. %, of tropolone, or a derivative thereof. In certain embodiments, the sunscreen base may comprise 10, 8, 7, 6, 5, 4, 3, or 2 wt. % or less; or 0.1, 0.2, 0.3, 0.4, 0.5, 1, 2, 3, 4, 5, 6, 8 wt. % or more tropolone, or a derivative thereof. In certain specific embodiments, the sunscreen base comprises from about 0.2 wt. % to about 5 wt. % tropolone, or a derivative thereof.

In certain embodiments, the sunscreen base comprises hinokitiol. Hinokitiol (2-hydroxy-6-(propan-2-yl)cyclohepta-2,4,6-trien-1-one) is a monoterpene found in the wood of trees in the family Cupressaceae. The sunscreen base may comprise from about 0.05 wt. % to about 15 wt. % hinokitiol (2-hydroxy-6-(propan-2-yl)cyclohepta-2,4,6-trien-1-one), or from about 0.05 wt. % to about 10 wt. %, about 0.05 wt. % to about 5 wt. %, about 0.1 wt. % to about 3 wt. %, about 0.1 wt. % to about 2 wt. %, about 0.1 wt. % to about 15 wt. %, about 0.1 wt. % to about 10 wt. %, about 0.1 wt. % to about 1 wt. %, about 0.1 wt. % to about 0.5 wt. %, or about 0.1 wt. % to about 0.3 wt. % hinokitiol (2-hydroxy-6-(propan-2-yl)cyclohepta-2,4,6-trien-1-one). It may comprise, for example, about 0.05, 0.1, 0.2, 0.3, 0.4, 0.5, 1, 2, 3, 4, 4.5, 5, 5.5, 6, 7, 7.5, 8, 9, 10, 11, 12, or 15 wt. % hinokitiol (2-hydroxy-6-(propan-2-yl)cyclohepta-2,4,6-trien-1-one). In certain embodiments, the sunscreen base comprises from about 0.05 wt. % to about 5 wt. %, optionally from about 0.1 wt. % to about 1 wt. %, of hinokitiol (2-hydroxy-6-(propan-2-yl)cyclohepta-2,4,6-trien-1-one). In certain embodiments, the sunscreen base may comprise 10, 8, 7, 6, 5, 4, 3, or 2 wt. % or less; or 0.1, 0.2, 0.3, 0.4, 0.5, 1, 2, 3, 4, 5, 6, 8 wt. % or more hinokitiol (2-hydroxy-6-(propan-2-yl)cyclohepta-2,4,6-trien-1-one). In certain specific embodiments, the sunscreen base comprises from about 0.2 wt. % to about 5 wt. % hinokitiol.

The sunscreen base may comprise from about 0.1 wt. % to about 65 wt. % of the carrier oil, or from about 0.1 wt. % to about 50 wt. %, about 0.1 wt. % to about 30 wt. %, about 0.5 wt. % to about 50 wt. %, about 0.5 wt. % to about 30 wt. %, about 0.5 wt. % to about 15 wt. %, about 0.5 wt. % to about 10 wt. %, about 1 wt. % to about 10 wt. %, about 2 wt. % to about 10 wt. %, or about 2 wt. % to about 7 wt. % of the carrier oil. It may comprise, for example, about 0.1, 0.2, 0.5, 1, 2, 3, 4, 4.5, 5, 5.5, 6, 7, 7.5, 8, 9, 10, 11, 12, 15, 20, 25, 30, 35, 40, 45, 50, 55, 60, or 65 wt. % of the carrier oil. In certain embodiments, the sunscreen base comprises from about 5 wt. % to about 50 wt. % of the carrier oil. The carrier oil may be selected from carrier oils known in the art. In certain embodiments, it may include plant derived carrier oils. It may, for example, be selected from the group consisting of jojoba, sesame, almond, and other plant oils, cetyl alcohol, triethylhexanoin, and fatty acids. In certain embodiments, the carrier oil is selected from the group consisting of Helianthus annuus (sunflower) seed oil, Simmondsia chinensis (jojoba) oil, and combinations thereof. In certain embodiments, the sunscreen base comprises from about 10 wt. % to about 35 wt. % of the carrier oil. In certain specific embodiments, the sunscreen base comprises from about 15 wt. % to about 50 wt. % of the carrier oil. In certain specific embodiments, the carrier oil comprises Helianthus annuus seed oil.

The dispersing and/or wetting agent may be an agent that can be added to a suspension of solid or liquid particles in a liquid to improve the separation of the particles and to prevent their settling or clumping. The sunscreen base may comprise from about 0.1 wt. % to about 15 wt. % of the dispersing and/or wetting agent, or from about 0.1 wt. % to about 10 wt. %, about 1 wt. % to about 10 wt. %, about 5 wt. % to about 15 wt. %, about 5 wt. % to about 10 wt. %, about 0.5 wt. % to about 15 wt. %, about 0.5 wt. % to about 10 wt. %, about 1 wt. % to about 10 wt. %, about 2 wt. % to about 10 wt. %, or about 2 wt. % to about 7 wt. % of the dispersing and/or wetting agent. It may comprise, for example, about 0.1, 0.2, 0.5, 1, 2, 3, 4, 4.5, 5, 5.5, 6, 7, 7.5, 8, 9, 10, 11, 12, or 15 wt. % of the dispersing and/or wetting agent. In certain embodiments, the sunscreen base comprises from about 1 wt. % to about 15 wt. % of the dispersing and/or wetting agent. The dispersing and/or wetting agent may be selected from the group consisting of cetearyl alcohol, stearic acid, glyceryl stearate, hydroxystearic acid, polyhydroxystearic acid, sodium polyaspartate, sodium polyacrylate, butyl PVP (polyvinyl pyrrolidone), sodium polynaphthalene sulfonate, isostearic acid, potassium cetyl phosphate, and ceteareth-20. In certain embodiments, the dispersing and/or wetting agent is selected from the group consisting of hydroxystearic acid, isostearic acid, potassium cetyl phosphate, and combinations thereof. In certain specific embodiments, the sunscreen base comprises from about 2.5 wt. % to about 10 wt. % of the emulsifier. In certain specific embodiments, the dispersing and/or wetting agent comprises isostearic acid. In certain specific embodiments, the sunscreen base comprises from about 0.2 wt. % to about 2 wt. % of the dispersing and/or wetting agent.

In certain embodiments, the sunscreen base further comprises an emulsifier. The emulsifier may be an agent that can stabilise the formation of an emulsion. In certain embodiments an agent may act as both a dispersing and/or wetting agent and an emulsifier. The sunscreen base may comprise from about 0.1 wt. % to about 15 wt. % of the emulsifier, or from about 0.1 wt. % to about 10 wt. %, about 1 wt. % to about 10 wt. %, about 5 wt. % to about 15 wt. %, about 5 wt. % to about 10 wt. %, about 0.5 wt. % to about 15 wt. %, about 0.5 wt. % to about 10 wt. %, about 1 wt. % to about 10 wt. %, about 2 wt. % to about 10 wt. %, or about 2 wt. % to about 7 wt. % of the emulsifier. It may comprise, for example, about 0.1, 0.2, 0.5, 1, 2, 3, 4, 4.5, 5, 5.5, 6, 7, 7.5, 8, 9, 10, 11, 12, or 15 wt. % of the emulsifier. In certain embodiments, the sunscreen base comprises from about 1 wt. % to about 15 wt. % of the emulsifier. The emulsifier may be selected from the group consisting of cetearyl alcohol, stearic acid, glyceryl stearate, hydroxystearic acid, polyhydroxystearic acid, butyl PVP (polyvinyl pyrrolidone), sodium polynaphthalene sulfonate, isostearic acid, potassium cetyl phosphate, and ceteareth-20. In certain embodiments, the emulsifier is selected from the group consisting of hydroxystearic acid, isostearic acid, potassium cetyl phosphate, and combinations thereof. In certain specific embodiments, the sunscreen base comprises from about 2.5 wt. % to about 10 wt. % of the emulsifier. In certain specific embodiments, the emulsifier comprises polyglyceryl-3 polyricinoleate.

The sunscreen base may comprise from about 0.1 wt. % to about 60 wt. % of the calcinated mixture, or from about 0.1 wt. % to about 40 wt. %, about 0.1 wt. % to about 30 wt. %, about 0.5 wt. % to about 50 wt. %, about 0.5 wt. % to about 30 wt. %, about 10 wt. % to about 30 wt. %, about 20 wt. % to about 30 wt. %, about 25 wt. % to about 35 wt. %, about 25 wt. % to about 30 wt. %, or about 27 wt. % to about 29 wt. % of the calcinated mixture. It may comprise, for example, about 0.1, 0.2, 0.5, 1, 2, 3, 4, 4.5, 5, 5.5, 6, 7, 7.5, 8, 9, 10, 11, 12, 15, 20, 25, 26, 27, 28, 29, 30, 35, 40, 45, 50 or 60 wt. % of the calcinated mixture. In certain specific embodiments, the sunscreen base comprises from about 10 wt. % to about 50 wt. % of the calcinated mixture.

In certain embodiments, the sunscreen base further comprises pristine zinc oxide. As used herein, the term “pristine zinc oxide” means a zinc oxide which is substantially pure, and is not a component of a calcinated mixture of zinc oxide and aluminium oxide. The sunscreen base may comprise from about 0.1 wt. % to about 25 wt. % of the pristine zinc oxide, or from about 0.1 wt. % to about 20 wt. %, about 0.1 wt. % to about 10 wt. %, about 0.5 wt. % to about 10 wt. %, about 0.5 wt. % to about 6 wt. %, about 2 wt. % to about 15 wt. %, about 2 wt. % to about 10 wt. %, about 3 wt. % to about 10 wt. %, about 4 wt. % to about 10 wt. %, or about 3 wt. % to about 5 wt. % of the pristine zinc oxide. It may comprise, for example, about 0.1, 0.2, 0.5, 1, 2, 3, 4, 4.5, 5, 5.5, 6, 7, 7.5, 8, 9, 10, 11, 12, 15, 20, or wt. % of the pristine zinc oxide. In certain embodiments, the sunscreen base comprises from about 1 wt. % to about 10 wt. % of the pristine zinc oxide. In certain embodiments, the sunscreen base comprises from about 10 wt. % to about 50 wt. % of the pristine zinc oxide.

In a second aspect of the invention there is provided a sunscreen composition, comprising:
a calcinated mixture comprising aluminium oxide and zinc oxide;
an emulsifier;
a dispersing and/or wetting agent;
a carrier oil; and
an aqueous phase.

The following options may be used in conjunction with the second aspect, either individually or in any combination.

In certain embodiments, the sunscreen composition comprises tropolone, or a derivative thereof. In this context, the tropolone derivative may be as hereinbefore described with respect to the first aspect. In certain embodiments, the sunscreen composition comprises from about 0.05 wt. % to about 15 wt. % tropolone, or a derivative thereof, or from about 0.05 wt. % to about 10 wt. %, about 0.05 wt. % to about 5 wt. %, about 0.1 wt. % to about 3 wt. %, about 0.1 wt. % to about 2 wt. %, about 0.1 wt. % to about 15 wt. %, about 0.1 wt. % to about 10 wt. %, about 0.1 wt. % to about 1 wt. %, about 0.1 wt. % to about 0.5 wt. %, or about 0.1 wt. % to about 0.3 wt. % tropolone, or a derivative thereof. It may comprise, for example, about 0.05, 0.1, 0.2, 0.3, 0.4, 0.5, 1, 2, 3, 4, 4.5, 5, 5.5, 6, 7, 7.5, 8, 9, 10, 11, 12, or 15 wt. % tropolone, or a derivative thereof. In certain embodiments, the sunscreen composition comprises from about 0.05 wt. % to about 5 wt. %, optionally from about 0.1 wt. % to about 1 wt. %, of tropolone, or a derivative thereof. In certain embodiments, the sunscreen composition may comprise 10, 8, 7, 6, 5, 4, 3, or 2 wt. % or less; or 0.1, 0.2, 0.3, 0.4, 0.5, 1, 2, 3, 4, 5, 6, 8 wt. % or more tropolone, or a derivative thereof. In certain specific embodiments, the sunscreen composition comprises from about 0.2 wt. % to about 5 wt. % tropolone, or a derivative thereof.

In certain embodiments, the sunscreen composition comprises hinokitiol. The sunscreen composition may comprise from about 0.05 wt. % to about 15 wt. % hinokitiol (2-hydroxy-6-(propan-2-yl)cyclohepta-2,4,6-trien-1-one), or from about 0.05 wt. % to about 10 wt. %, about 0.05 wt. % to about 5 wt. %, about 0.1 wt. % to about 3 wt. %, about 0.1 wt. % to about 2 wt. %, about 0.1 wt. % to about 15 wt. %, about 0.1 wt. % to about 10 wt. %, about 0.1 wt. % to about 1 wt. %, about 0.1 wt. % to about 0.5 wt. %, or about 0.1 wt. % to about 0.3 wt. % hinokitiol (2-hydroxy-6-(propan-2-yl)cyclohepta-2,4,6-trien-1-one). It may comprise, for example, about 0.05, 0.1, 0.2, 0.3, 0.4, 0.5, 1, 2, 3, 4, 4.5, 5, 5.5, 6, 7, 7.5, 8, 9, 10, 11, 12, or 15 wt. % hinokitiol (2-hydroxy-6-(propan-2-yl)cyclohepta-2,4,6-trien-1-one). In certain embodiments, the sunscreen composition comprises from about 0.05 wt. % to about 5 wt. %, optionally from about 0.1 wt. % to about 1 wt. %, of hinokitiol (2-hydroxy-6-(propan-2-yl)cyclohepta-2,4,6-trien-1-one). In certain embodiments, the sunscreen composition may comprise 10, 8, 7, 6, 5, 4, 3, or 2 wt. % or less; or 0.1, 0.2, 0.3, 0.4, 0.5, 1, 2, 3, 4, 5, 6, 8 wt. % or more hinokitiol (2-hydroxy-6-(propan-2-yl)cyclohepta-2,4,6-trien-1-one). In certain specific embodiments, the sunscreen composition comprises from about 0.1 wt. % to about 2.5 wt. % hinokitiol.

The sunscreen composition may comprise from about 2 wt. % to about 60 wt. % of the calcinated mixture, or from about 2 wt. % to about 40 wt. %, about 2 wt. % to about 30 wt. %, about 2 wt. % to about 25 wt. %, about 2 wt. % to about 50 wt. %, about 2 wt. % to about 30 wt. %, about 10 wt. % to about 30 wt. %, about 20 wt. % to about 30 wt. %, about 25 wt. % to about 35 wt. %, about 25 wt. % to about 30 wt. %, or about 27 wt. % to about 29 wt. % of the calcinated mixture. It may comprise, for example, about 2, 3, 4, 4.5, 5, 5.5, 6, 7, 7.5, 8, 9, 10, 11, 12, 15, 20, 25, 26, 27, 28, 29, 30, 35, 40, 45, 50 or 60 wt. % of the calcinated mixture. In certain embodiments, the sunscreen composition comprises from about 5 wt. % to about 30 wt. % of the calcinated mixture.

The sunscreen composition may comprise from about 10 wt. % to about 65 wt. % of the carrier oil, or from about 10 wt. % to about 60 wt. %, about 10 wt. % to about 50 wt. %, about 10 wt. % to about 60 wt. %, about 10 wt. % to about 30 wt. %, about 15 wt. % to about 45 wt. %, about 15 wt. % to about 60 wt. %, about 20 wt. % to about 60 wt. %, about 10 wt. % to about 20 wt. %, or about 20 wt. % to about 30 wt. % of the carrier oil. It may comprise, for example, about 10, 11, 12, 15, 20, 25, 30, 35, 40, 45, 50, 55, 60, or 65 wt. % of the carrier oil. In certain embodiments, the sunscreen composition comprises from about 5 wt. % to about 20 wt. % of the carrier oil. The carrier oil may be selected from carrier oils known in the art. In certain embodiments, it may include plant derived oils. It may, for example, be selected from the group consisting of mineral oil, jojoba, sesame, almond, and other plant oils, cetyl alcohol, triethylhexanoin, and fatty acids. In certain embodiments, the carrier oil is selected from the group consisting of Helianthus annuus (sunflower) seed oil, Simmondsia chinensis (jojoba) oil, and combinations thereof. In certain embodiments, the sunscreen composition comprises from about 10 wt. % to about 35 wt. % of the carrier oil.

The aqueous phase may comprise pure, or substantially pure water. It may comprise one or more salts or water-soluble organic compounds dissolved therein. It may comprise water, one or more salts, one or more preservatives, one or more humectants, and/or one or more polymers. The sunscreen composition may comprise from about 0.1 wt. % to about 50 wt. % of the aqueous phase, or from about 0.1 wt. % to about 40 wt. %, about 0.1 wt. % to about 30 wt. %, about 0.5 wt. % to about 50 wt. %, about 0.5 wt. % to about 30 wt. %, about 0.5 wt. % to about 25 wt. %, about 0.5 wt. % to about 25 wt. %, about 10 wt. % to about 25 wt. %, about 20 wt. % to about 25 wt. %, or about 21 wt. % to about 23 wt. % of the aqueous phase. It may comprise, for example, about 0.1, 0.2, 0.5, 1, 2, 3, 4, 4.5, 5, 5.5, 6, 7, 7.5, 8, 9, 10, 11, 12, 15, 20, 21, 22, 23, 24, 25, 30, 35, 40, 45, or 50 wt. % of the aqueous phase. In certain embodiments, the sunscreen composition comprises from about 5 wt. % to about 50 wt. % of the aqueous phase. In certain specific embodiments, the sunscreen composition comprises from about 20 wt. % to about 40 wt. % of the aqueous phase.

The dispersing and/or wetting agent may be an agent that can be added to a suspension of solid or liquid particles in a liquid to improve the separation of the particles and to prevent their settling or clumping. The sunscreen composition may comprise from about 0.1 wt. % to about 15 wt. % of the dispersing and/or wetting agent, or from about 0.1 wt. % to about 10 wt. %, about 1 wt. % to about 10 wt. %, about 5 wt. % to about 15 wt. %, about 5 wt. % to about 10 wt. %, about 0.5 wt. % to about 15 wt. %, about 0.5 wt. % to about 10 wt. %, about 1 wt. % to about 10 wt. %, about 2 wt. % to about 10 wt. %, or about 2 wt. % to about 7 wt. % of the dispersing and/or wetting agent. It may comprise, for example, about 0.1, 0.2, 0.5, 1, 2, 3, 4, 4.5, 5, 5.5, 6, 7, 7.5, 8, 9, 10, 11, 12, or 15 wt. % of the dispersing and/or wetting agent. In certain embodiments, the sunscreen composition comprises from about 1 wt. % to about 15 wt. % of the dispersing and/or wetting agent. The dispersing and/or wetting agent may be selected from the group consisting of cetearyl alcohol, stearic acid, glyceryl stearate, hydroxystearic acid, polyhydroxystearic acid, sodium polyaspartate, sodium polyacrylate, butyl PVP (polyvinyl pyrrolidone), sodium polynaphthalene sulfonate, isostearic acid, potassium cetyl phosphate, and ceteareth-20. In certain embodiments, the dispersing and/or wetting agent is selected from the group consisting of hydroxystearic acid, isostearic acid, potassium cetyl phosphate, and combinations thereof. In certain specific embodiments, the sunscreen composition comprises from about 2.5 wt. % to about 10 wt. % of the emulsifier. In certain specific embodiments, the dispersing and/or wetting agent comprises isostearic acid. In certain specific embodiments, the sunscreen composition comprises from about 0.2 wt. % to about 2 wt. % of the dispersing and/or wetting agent. In certain embodiments, the sunscreen composition comprises from about 0.2 wt. % to about 1.5 wt. % of the dispersing and/or wetting agent.

The emulsifier may be an agent that can stabilise the formation of an emulsion. In certain embodiments an agent may act as both a dispersing and/or wetting agent and an emulsifier. In certain embodiments, the emulsifier may be a polymer. The sunscreen composition may comprise from about 0.1 wt. % to about 15 wt. % of the emulsifier, or from about 0.1 wt. % to about 10 wt. %, about 1 wt. % to about 10 wt. %, about 5 wt. % to about 15 wt. %, about 5 wt. % to about 10 wt. %, about 0.5 wt. % to about 15 wt. %, about 0.5 wt. % to about 10 wt. %, about 1 wt. % to about 10 wt. %, about 2 wt. % to about 10 wt. %, or about 2 wt. % to about 7 wt. % of the emulsifier. It may comprise, for example, about 0.1, 0.2, 0.5, 1, 2, 3, 4, 4.5, 5, 5.5, 6, 7, 7.5, 8, 9, 10, 11, 12, or 15 wt. % of the emulsifier. In certain embodiments, the sunscreen composition comprises from about 1 wt. % to about 15 wt. % of the emulsifier. The emulsifier may be selected from the group consisting of cetearyl alcohol, stearic acid, glyceryl stearate, hydroxystearic acid, polyhydroxystearic acid, butyl PVP (polyvinyl pyrrolidone), sodium polynaphthalene sulfonate, isostearic acid, potassium cetyl phosphate, and ceteareth-20. In certain embodiments, the emulsifier is selected from the group consisting of hydroxystearic acid, isostearic acid, potassium cetyl phosphate, and combinations thereof. In certain specific embodiments, the sunscreen composition comprises from about 2.5 wt. % to about 10 wt. % of the emulsifier. In certain specific embodiments, the emulsifier comprises polyglyceryl-3 polyricinoleate. In certain embodiments, the sunscreen composition comprises from about 2 wt. % to about 5 wt. % of the emulsifier.

In certain embodiments, the sunscreen composition comprises a water-in-oil emulsion.

In certain embodiments, the sunscreen composition comprises from 0.01 wt % to 10 wt % hinokitiol, or from 0.1 wt % to 5 wt % hinokitiol, or from 0.1 to 4 wt %, or from 0.1 to 3 wt %, or form 0.1 to 2 wt %, or from 0.1 to 1 wt %, or from 0.2 to 0.9 wt %, or from 0.25 to 0.8 wt %, or from 0.3 to 0.7 wt %, or from 0.4 to 0.6 wt % or about 0.5 wt % hinokitiol.

In certain embodiments, the sunscreen composition may comprise one or more waxes. The one or more waxes may, for example, be selected from candelilla wax, cetyl alcohol and beeswax.

In certain embodiments, the carrier oil comprises one or more oils selected from vegetable oils, or one or more esters of organic acids derived from vegetable sources, or coco-caprylate/caprate. In one embodiment, the carrier oil includes coco-caprylate/caprate. In another embodiment, the carrier oil includes ethyl palmitate. In another embodiment, the carrier oil includes a vegetable oil, such as sunflower oil.

In some embodiments, the sunscreen composition is free from silicone materials. In some embodiments, the sunscreen composition is free from silanes or silane-based materials.

In one embodiment, the aqueous phase comprises water and other components that are soluble in water. The aqueous phase may include glycerin (glycerol) or other water soluble humectants, and one or more salts (such as NaCl). Other components may also be added to the aqueous phase. The components added to the aqueous phase may be water soluble components.

In one embodiment, the carrier oil may be a component of an oil phase. The oil phase may comprise one or more different compounds. Examples of compounds that may be included in the oil phase include one or more of oils, fatty acids, beeswax, phospholipids and/or glycerophospholipids, hydroxystearic acids, polyhydroxystearic acids, esters of fatty acids, polyesters of fatty acids, emulsifiers and emollients/moisturizers.

The zinc oxide that is present in the composition may be in the form of fine particles. The zinc oxide may comprise the product sold by Antaria Pty Ltd under the trade name ZinClear® XP and/or Zinclear® XP dispersions, e.g. Zinclear® XP65 COCO. These products may be as described in Australian patent number 2009203996 and in international patent publication number WO 2009/089523, the entire contents of which are herein incorporated by cross-reference. The zinc oxide particles may also be the same as the zinc oxide particles used in the sunscreen composition described in Australian patent number 2003205436, the entire contents of which are herein incorporated by cross-reference. ZinClear® XP65 COCO is a 65 wt % dispersion of zinc oxide in coco-caprylate/caprate. Coco-Caprylate/Caprate is a straight, unbranched wax ester made of C₁₂-C₁₈ coconut fatty alcohol and a defined blend of fractionated fatty acids of vegetable origin, commonly used in cosmetic products.

In one embodiment, the zinc oxide comprises Zinclear® XP65 COCO and ZinClear® XP.

In some embodiments, the zinc oxide may be in the form of aggregates of primary particles.

ZinClear® XP comprises zinc oxide particles that are formed as aggregates of smaller particles. As described in Australian patent number 2009203996, ZinClear® XP is a zinc oxide powder comprising zinc oxide agglomerates which, when used in a dispersion at a concentration of 50 weight % of zinc oxide, produces a transparent composition having a total visible transmittance through a path length of 20 μm at 550 nm of at least 70%, the powder having a number average zinc oxide aggregate size of at least 0.8 μm, wherein the aggregates are mesoporous and have a total mesopore volume of at least 0.25 cm3 per gram. High-energy milling of ZinClear® XP can break up the zinc oxide aggregates to reduce the overall particle size of the aggregates. ZinClear® XP comprises 99 to 100% zinc oxide.

Zinclear® XP65 COCO is a mixture of zinc oxide (in the form of ZinClear® XP), coco-caprylate/caprate, polygylceryl-3 polyricinoleate and isostearic acid. It is a commercially available product can be purchased from Antaria Pty Ltd. Zinclear® XP65 COCO is a dispersion that includes coco-caprylate/caprate as an emollient. It has high transparency and low whiteness. Zinclear® XP65 COCO contains 65% zinc oxide, 30 to 31% coco-caprylate/caprate, 3.0-3.5% polygylceryl-3 polyricinoleate and 1 to 1.5% isostearic acid.

In another embodiment, the zinc oxide comprises ZinClear® XP.

In one embodiment, the sunscreen composition is free of titanium dioxide.

In one embodiment, the water phase may comprise water, glycerin, sodium chloride and galactoarabinan. Glycerin and galactoarabinan are moisturizers and they may also affect the film forming capabilities of the composition.

The sunscreen composition, in some embodiments, may also contain other ingredients, such as preservatives and fragrances. Chelators and other ingredients which may boost the efficiency of preservatives may also be added.

In one embodiment, the sunscreen composition contains no anti-inflammatory active agents.

In certain embodiments, the sunscreen composition further comprises pristine zinc oxide. The sunscreen composition may comprise from about 0.1 wt. % to about 25 wt. % of the pristine zinc oxide, or from about 0.1 wt. % to about 20 wt. %, about 0.1 wt. % to about 10 wt. %, about 0.5 wt. % to about 10 wt. %, about 0.5 wt. % to about 6 wt. %, about 2 wt. % to about 15 wt. %, about 2 wt. % to about 10 wt. %, about 3 wt. % to about 10 wt. %, about 4 wt. % to about 10 wt. %, or about 3 wt. % to about 5 wt. % of the pristine zinc oxide. It may comprise, for example, about 0.1, 0.2, 0.5, 1, 2, 3, 4, 4.5, 5, 5.5, 6, 7, 7.5, 8, 9, 10, 11, 12, 15, 20, or 25 wt. % of the pristine zinc oxide. In certain embodiments, the sunscreen composition comprises from about 1 wt. % to about 10 wt. % of the pristine zinc oxide. In certain embodiments, the sunscreen composition comprises from about 10 wt. % to about 50 wt. %, optionally from about 10 wt. % to about 25 wt. % of the pristine zinc oxide. In certain embodiments, the sunscreen composition comprises from about 5 wt. % to about 40 wt. %, optionally from about 5 wt. % to about 25 wt. % of the pristine zinc oxide.

In certain embodiments, the sunscreen composition further comprises one or more additional components selected from the group consisting of humectants, salts, chelating agents, stabilizing agents, texturizing agents, emollients, film forming agents, occlusive agents, and water-resistant agents.

The emollient may be an agent or agents that are capable of softening or soothing skin. In certain embodiments, the carrier oil may itself be an emollient. The sunscreen composition may comprise from about 0.1 wt. % to about 65 wt. % of the emollient, or from about 0.1 wt. % to about 50 wt. %, about 0.1 wt. % to about 30 wt. %, about 0.5 wt. % to about 50 wt. %, about 0.5 wt. % to about 30 wt. %, about 0.5 wt. % to about 15 wt. %, about 0.5 wt. % to about 10 wt. %, about 1 wt. % to about 10 wt. %, about 2 wt. % to about 10 wt. %, or about 2 wt. % to about 7 wt. % of the emollient. It may comprise, for example, about 0.1, 0.2, 0.5, 1, 2, 3, 4, 4.5, 5, 5.5, 6, 7, 7.5, 8, 9, 10, 11, 12, 15, 20, 25, 30, 35, 40, 45, 50, 55, 60, or 65 wt. % of the emollient. In certain embodiments, the sunscreen composition comprises from about 5 wt. % to about 20 wt. % of the emollient. The emollient may be selected from emollients known in the art. It may, for example, be selected from the group consisting of shea butter, cocoa butter, lanolin, paraffin, beeswax, squalene, coconut, jojoba, sesame, almond, and other plant oils, cetyl alcohol, triethylhexanoin, and fatty acids. In certain embodiments, the emollient is selected from the group consisting of Helianthus annuus (sunflower) seed oil, Simmondsia chinensis (jojoba) oil, and combinations thereof.

The humectant may be a hygroscopic substance capable of retaining moisture. The sunscreen composition may comprise from about 0.1 wt. % to about 20 wt. % of the humectant, or from about 0.1 wt. % to about 10 wt. %, about 1 wt. % to about 10 wt. %, about 0.5 wt. % to about 20 wt. %, about 0.5 wt. % to about 10 wt. %, about 0.5 wt. % to about 15 wt. %, about 0.5 wt. % to about 8 wt. %, about 3 wt. % to about 10 wt. %, about 2 wt. % to about 10 wt. %, or about 7 wt. % to about 12 wt. % of the humectant. It may comprise, for example, about 0.1, 0.2, 0.5, 1, 2, 3, 4, 4.5, 5, 5.5, 6, 7, 7.5, 8, 9, 10, 11, 12, 15, or 20 wt. % of the humectant. In certain embodiments, the sunscreen composition comprises from about 1 wt. % to about 20 wt. % of the humectant. The humectant may be selected from the group consisting of hyaluronic acid, propylene glycol, glycerin, panthenol, and polyglutamic acid. In certain embodiments, the humectant comprises glycerin.

In certain embodiments, the sunscreen composition further comprises one or more salts. The weight percentage of salts in the sunscreen composition may be may be from about 0.1 wt. % to about 5 wt. %, or from about 0.11 wt. % to about 5 wt. %, about 0.12 wt. % to about 5 wt. %, about 0.14 wt. % to about 5 wt. %, about 0.18 wt. % to about 5 wt. %, about 0.22 wt. % to about 5 wt. %, about 0.26 wt. % to about 5 wt. %, about 0.3 wt. % to about 5 wt. %, about 0.34 wt. % to about 5 wt. %, about 0.38 wt. % to about 5 wt. %, about 0.42 wt. % to about 5 wt. %, about 0.46 wt. % to about 5 wt. %, about 0.5 wt. % to about 5 wt. %, about 0.1 wt. % to about 5 wt. %, about 0.1 wt. % to about 5 wt. %, about 0.1 wt. % to about 5 wt. %, about 0.1 wt. % to about 5 wt. %, about 0.5 wt. % to about 5 wt. %, about 2.4 wt. % to about 5 wt. %, about 4.3 wt. % to about 5 wt. %, about 0.5 wt. % to about 4.3 wt. %, or about 0.5 wt. % to about 2.4 wt. %, It may be greater than or equal to about 0.1 wt. %, 0.11 wt. %, 0.12 wt. %, 0.14 wt. %, 0.18 wt. %, 0.22 wt. %, 0.26 wt. %, 0.3 wt. %, 0.34 wt. %, 0.38 wt. %, 0.42 wt. %, 0.46 wt. %, 0.5 wt. %, 1 wt. %, 2 wt. %, 3 wt. %, or 4 wt. %, It may be less than or equal to about 5 wt. %, 4 wt. %, 3 wt. %, 2 wt. %, or 1 wt. %, In certain embodiments, it may be, for example, about 0.1 wt. %, 0.11 wt. %, 0.12 wt. %, 0.14 wt. %, 0.18 wt. %, 0.22 wt. %, 0.26 wt. %, 0.3 wt. %, 0.34 wt. %, 0.38 wt. %, 0.42 wt. %, 0.46 wt. %, 0.5 wt. %, 1 wt. %, 1.4 wt. %, 1.9 wt. %, 2.4 wt. %, 2.9 wt. %, 3.4 wt. %, 3.8 wt. %, 4.3 wt. %, 4.8 wt. %, or 5 wt. %.

In certain embodiments, the sunscreen composition further comprises one or more occlusive agents. The weight percentage of occlusive agents in the sunscreen composition may be may be from about 0.1 wt. % to about 20 wt. %, or from about 0.11 wt. % to about 20 wt. %, about 0.12 wt. % to about 20 wt. %, about 0.14 wt. % to about 20 wt. %, about 0.18 wt. % to about 20 wt. %, about 0.22 wt. % to about 20 wt. %, about 0.26 wt. % to about 20 wt. %, about 0.3 wt. % to about 20 wt. %, about 0.34 wt. % to about 20 wt. %, about 0.38 wt. % to about 20 wt. %, about 0.42 wt. % to about 20 wt. %, about 0.46 wt. % to about 20 wt. %, about 0.5 wt. % to about 20 wt. %, about 0.1 wt. % to about 19 wt. %, about 0.1 wt. % to about 18 wt. %, about 0.1 wt. % to about 17 wt. %, about 0.1 wt. % to about 16 wt. %, about 0.1 wt. % to about 15 wt. %, about 0.1 wt. % to about 14 wt. %, about 0.1 wt. % to about 13 wt. %, about 0.1 wt. % to about 12 wt. %, about 0.1 wt. % to about 11 wt. %, about 0.1 wt. % to about 10 wt. %, about 0.5 wt. % to about 10 wt. %, about 2.4 wt. % to about 10 wt. %, about 4.3 wt. % to about 10 wt. %, about 6.2 wt. % to about 10 wt. %, about 8.1 wt. % to about 10 wt. %, about 0.5 wt. % to about 8.1 wt. %, about 0.5 wt. % to about 6.2 wt. %, about 0.5 wt. % to about 4.3 wt. %, or about 0.5 wt. % to about 2.4 wt. %, It may be greater than or equal to about 0.1 wt. %, 0.11 wt. %, 0.12 wt. %, 0.14 wt. %, 0.18 wt. %, 0.22 wt. %, 0.26 wt. %, 0.3 wt. %, 0.34 wt. %, 0.38 wt. %, 0.42 wt. %, 0.46 wt. %, 0.5 wt. %, 1 wt. %, 2 wt. %, 3 wt. %, or 4 wt. %, It may be less than or equal to about 20 wt. %, 19 wt. %, 18 wt. %, 17 wt. %, 16 wt. %, 15 wt. %, 14 wt. %, 13 wt. %, 12 wt. %, 11 wt. %, 10 wt. %, 9 wt. %, 8 wt. %, 7 wt. %, or 6 wt. %, In certain embodiments, it may be, for example, about 0.1 wt. %, 0.11 wt. %, 0.12 wt. %, 0.14 wt. %, 0.18 wt. %, 0.22 wt. %, 0.26 wt. %, 0.3 wt. %, 0.34 wt. %, 0.38 wt. %, 0.42 wt. %, 0.46 wt. %, 0.5 wt. %, 1 wt. %, 1.4 wt. %, 1.9 wt. %, 2.4 wt. %, 2.9 wt. %, 3.4 wt. %, 3.8 wt. %, 4.3 wt. %, 4.8 wt. %, 5.2 wt. %, 5.7 wt. %, 6.2 wt. %, 6.7 wt. %, 7.2 wt. %, 7.6 wt. %, 8.1 wt. %, 8.6 wt. %, 9 wt. %, 9.5 wt. %, 10 wt. %, 12 wt. %, 13 wt. %, 14 wt. %, 15 wt. %, 16 wt. %, 17 wt. %, 18 wt. %, 19 wt. %, or 20 wt. %.

In certain embodiments, the sunscreen composition further comprises one or more water resistant agents. The weight percentage of water resistant agents in the sunscreen composition may be may be from about 0.1 wt. % to about 20 wt. %, or from about 0.11 wt. % to about 20 wt. %, about 0.12 wt. % to about 20 wt. %, about 0.14 wt. % to about 20 wt. %, about 0.18 wt. % to about 20 wt. %, about 0.22 wt. % to about 20 wt. %, about 0.26 wt. % to about 20 wt. %, about 0.3 wt. % to about 20 wt. %, about 0.34 wt. % to about 20 wt. %, about 0.38 wt. % to about 20 wt. %, about 0.42 wt. % to about 20 wt. %, about 0.46 wt. % to about 20 wt. %, about 0.5 wt. % to about 20 wt. %, about 0.1 wt. % to about 19 wt. %, about 0.1 wt. % to about 18 wt. %, about 0.1 wt. % to about 17 wt. %, about 0.1 wt. % to about 16 wt. %, about 0.1 wt. % to about 15 wt. %, about 0.1 wt. % to about 14 wt. %, about 0.1 wt. % to about 13 wt. %, about 0.1 wt. % to about 12 wt. %, about 0.1 wt. % to about 11 wt. %, about 0.1 wt. % to about 10 wt. %, about 0.5 wt. % to about 10 wt. %, about 2.4 wt. % to about 10 wt. %, about 4.3 wt. % to about 10 wt. %, about 6.2 wt. % to about 10 wt. %, about 8.1 wt. % to about 10 wt. %, about 0.5 wt. % to about 8.1 wt. %, about 0.5 wt. % to about 6.2 wt. %, about 0.5 wt. % to about 4.3 wt. %, or about 0.5 wt. % to about 2.4 wt. %, It may be greater than or equal to about 0.1 wt. %, 0.11 wt. %, 0.12 wt. %, 0.14 wt. %, 0.18 wt. %, 0.22 wt. %, 0.26 wt. %, 0.3 wt. %, 0.34 wt. %, 0.38 wt. %, 0.42 wt. %, 0.46 wt. %, 0.5 wt. %, 1 wt. %, 2 wt. %, 3 wt. %, or 4 wt. %, It may be less than or equal to about 20 wt. %, 19 wt. %, 18 wt. %, 17 wt. %, 16 wt. %, 15 wt. %, 14 wt. %, 13 wt. %, 12 wt. %, 11 wt. %, 10 wt. %, 9 wt. %, 8 wt. %, 7 wt. %, or 6 wt. %, In certain embodiments, it may be, for example, about 0.1 wt. %, 0.11 wt. %, 0.12 wt. %, 0.14 wt. %, 0.18 wt. %, 0.22 wt. %, 0.26 wt. %, 0.3 wt. %, 0.34 wt. %, 0.38 wt. %, 0.42 wt. %, 0.46 wt. %, 0.5 wt. %, 1 wt. %, 1.4 wt. %, 1.9 wt. %, 2.4 wt. %, 2.9 wt. %, 3.4 wt. %, 3.8 wt. %, 4.3 wt. %, 4.8 wt. %, 5.2 wt. %, 5.7 wt. %, 6.2 wt. %, 6.7 wt. %, 7.2 wt. %, 7.6 wt. %, 8.1 wt. %, 8.6 wt. %, 9 wt. %, 9.5 wt. %, 10 wt. %, 12 wt. %, 13 wt. %, 14 wt. %, 15 wt. %, 16 wt. %, 17 wt. %, 18 wt. %, 19 wt. %, or 20 wt. %.

In certain embodiments, the sunscreen composition further comprises a viscosity adjusting agent. The viscosity adjusting agent may increase or decrease the viscosity of the formulation. The sunscreen composition may comprise from about 0.1 wt. % to about 10 wt. % of the viscosity adjusting agent, or from about 0.5 wt. % to about 10 wt. %, about 1 wt. % to about 10 wt. %, about 1 wt. % to about 9 wt. %, about 1 wt. % to about 8 wt. %, about 1 wt. % to about 5 wt. %, or about 1 wt. % to about 3 wt. % of the viscosity adjusting agent. It may comprise, for example, about 0.1, 0.2, 0.5, 1, 2, 3, 4, 4.5, 5, 5.5, 6, 7, 7.5, 8, 9, or 10 wt. % of the viscosity adjusting agent. In certain embodiments, the sunscreen composition comprises from about 0.1 wt. % to about 5 wt. % of the viscosity adjusting agent. The viscosity adjusting agent may comprise one or more selected from the group consisting of sodium chloride; propylene glycol, ethylene glycol and copolymers thereof; clays; and waxes. In certain embodiments, the viscosity adjusting agent comprises Euphorbia cerifera (candelilla wax).

In certain embodiments, the sunscreen composition further comprises a stabilising and/or texturizing agent. Stabilising agents may be agents which can maintain the function and activity of other agents in a formulation. Texturing agents may be agents which can enhance or improve the consistency and/or feel of finished products. In certain embodiments an agent may act as both a stabilising agent and a texturizing agent. The sunscreen composition may comprise from about 0.1 wt. % to about 15 wt. % of the stabilising and/or texturizing agent, or from about 0.1 wt. % to about 10 wt. %, about 0.1 wt. % to about 8 wt. %, about 0.5 wt. % to about 5 wt. %, about 0.5 wt. % to about 15 wt. %, about 1 wt. % to about 15 wt. %, about 1 wt. % to about 10 wt. %, about 1 wt. % to about 8 wt. %, about 1 wt. % to about 5 wt. %, or about 1 wt. % to about 3 wt. % of the stabilising and/or texturizing agent. It may comprise, for example, about 0.1, 0.2, 0.5, 1, 2, 3, 4, 4.5, 5, 5.5, 6, 7, 7.5, 8, 9, 10, 11, 12, or 15 wt. % of the stabilising and/or texturizing agent. In certain embodiments, the sunscreen composition comprises from about 0.1 wt. % to about 5 wt. % of the stabilising and/or texturizing agent. The stabilising and/or texturizing agent may be selected from the group consisting of electrolytes, waxes, polysaccharides (for example, cellulose derivates, such as carboxy methylcellulose), gums, clays (such as bentonites), polyacrylates, oils, and esters. In certain embodiments, the stabilising and/or texturizing agent is selected from the group consisting of xanthan gum, maltodextrin, and combinations thereof.

In certain embodiments of the first or second aspect, the dispersing and/or wetting agent comprises isostearic acid.

In certain embodiments of the first or second aspect, the carrier oil comprises Helianthus annuus seed oil.

Disclosed herein is a sunscreen composition comprising:
from about 10 wt. % to about 20 wt. % Helianthus annuus seed oil;
from about 2 wt. % to about 5 wt. % polyglyceryl-3 polyricinoleate;
from about 0.5 wt. % to about 3 wt. % isostearic acid;
from about 10 wt. % to about 20 wt. % calcinated aluminium and zinc oxide;
from about 0 wt. % to about 7 wt. % of coconut alkanes and cococaprylate/caprate;
from about 10 wt. % to about 30 wt. % pristine zinc oxide;
from about 1 wt. % to about 5 wt. % jojoba oil;
from about 0.1 wt. % to about 1 wt. % polyhydroxystearic acid;
from about 0 wt. % to about 1 wt. % acrylate octylacrylamide copolymer;
from about 1 wt. % to about 5 wt. % wax;
from about 0 wt. % to about 5 wt. % soya oil;
from about 0.1 wt. % to about 2 wt. % xanthan gum;
from about 20 wt. % to about 40 wt. % water;
from about 5 wt. % to about 15 wt. % glycerin;
from about 0.1 wt. % to about 5 wt. % maltodextrin;
from about 0.1 wt. % to about 5 wt. % potassium cetyl phosphate;
from about 0.1 wt. % to about 5 wt. % sodium chloride; and
from about 0.01 wt. % to about 1 wt. % disodium EDTA.

Disclosed herein is a sunscreen composition comprising the following components:

| Ingredient Name | wt. % Range |
|---|---|
| Calcinated mixture of Zinc Oxide and | 20-40 |
| Aluminium Oxide | |
| Helianthus Annuus Seed Oil | 15-25 |
| Simmondsia Chinensis (Jojoba) Seed oil | 2-6 |
| Polyglyceryl-3 Polyricinoleate | 1-5 |
| Polyhydroxy Stearic Acid | 0.5-5 |
| Wax | 1-5 |
| Isostearic Acid | 0.5-3 |
| Xanthan Gum | 0.5-2 |
| Sodium Chloride | 0.5-2 |
| Glycerin | 5-15 |
| Maltodextrin | 0.5-3 |
| Potassium Cetyl Phosphate | 0.5-3 |
| Water | Up to 100 |

The sunscreen composition according to the second aspect may incorporate the sunscreen base according to the first aspect. The sunscreen base according to the first aspect may be a component of the sunscreen composition according to the second aspect.

In a third aspect of the invention there is provided a sunscreen composition, comprising the following components:

| Ingredient Name | wt. % Range | |
|---|---|---|
| Calcinated mixture of Zinc Oxide and | 25 to | 40 |
| Aluminium Oxide | | |
| Sunflower Oil | 15 to | 25 |
| Jojoba oil | 2 to | 5 |
| PolyGlyceryl Polyricinoleate (PGPR) | 2 to | 5 |
| Polyhydroxystearic Acid | 0.5 to | 5 |
| Hinokitiol | 0.25 to | 2 |
| Wax | 0.25 to | 3 |
| Isostearic Acid | 0.5 to | 3 |
| Xanthan Gum | 0.1 to | 2 |
| Water | 20 to | 35 |
| Glycerin | 5 to | 15 |
| Maltodextrin | 0.5 to | 3 |
| Amphisol K | 0.1 to | 2 |
| Sodium Chloride | 0.1 to | 5 |
| Sodium Phytate | 0.1 to | 0.5 |

The following options may be used in conjunction with the third aspect, either individually or in any combination.

In certain embodiments of the first, second or third aspects, the calcinated mixture comprises an aluminium oxide-zinc oxide composite. In certain embodiments of the first, second, or third aspect, the calcinated mixture comprises an aluminium doped zinc oxide.

The aluminium doped zinc oxide or composite may comprise from about 0.5 at. % to about 10 at. % aluminium, or from about 0.5 at. % to about 5 at. %, about 0.5 at. % to about 2 at. %, about 0.5 at. % to about 1 at. %, about 1 at. % to about 10 at. %, about 1 at. % to about 5 at. %, about 1 at. % to about 2 at. %, or about 0.7 at. % to about 1 at. % aluminium. It may comprise, for example, about 0.5, 1, 1.5, 2, 2.5, 3, 4, 5, 6, 7, 8, 9, or 10 at. % aluminium.

The aluminium doped zinc oxide or composite may be mesoporous. It may have a total mesopore volume of from about 0.2 cm3/g to about 0.9 cm3/g, or from about 0.25 cm3/g to about 0.9 cm3/g, about 0.30 cm3/g to about 0.9 cm3/g, about 0.4 cm3/g to about 0.9 cm3/g, about 0.5 cm3/g to about 0.9 cm3/g, about 0.4 cm3/g to about 0.8 cm3/g, about 0.4 cm3/g to about 0.7 cm3/g, or about 0.4 cm3/g to about 0.6 cm3/g. In certain embodiments of the first, second, or third aspect, the aluminium doped zinc oxide or composite may have a total mesopore volume of at least about 0.2, 0.25, 0.3, 0.4, 0.45, 0.5, 0.55, 0.6, 0.65, 0.7, 0.75, 0.8, or 0.85 cm3/g. In certain embodiments of the first, second, or third aspect, the aluminium doped zinc oxide or composite may, for example, have a total mesopore volume of about 0.2, 0.25, 0.3, 0.35, 0.4, 0.45, 0.47, 0.49, 0.5, 0.51, 0.52, 0.55, 0.57, 0.6, 0.65, 0.7, 0.75, 0.8, 0.85, or 0.9 cm3/g. In certain embodiments of the first, second, or third aspect, the aluminium doped zinc oxide or composite has a total mesopore volume of at least about 0.25 cm3/g. The average pore size in the aluminium doped zinc oxide or composite may be from about 5 nm to about 150 nm, or it may be from about 5 nm to about 100 nm, about 10 nm to about 100 nm, about 20 nm to about 100 nm, about 5 nm to about 80 nm, about 5 nm to about 60 nm, about 20 nm to about 80 nm, about 20 nm to about 60 nm, or about 40 nm to about 60 nm. It may, for example, have an average pore size of about 5, 10, 11, 12, 15, 20, 30, 40, 50, 55, 60, 65, 70, 80, 90, 100, 110, 120, 130, 140, or 150 nm.

In certain embodiments of the first, second, or third aspect, the aluminium doped zinc oxide or composite may have a specific surface area of from about 10 m²/g to about 100 m²/g, or it may be from about 10 m²/g to about 80 m²/g, about 10 m²/g to about 50 m²/g, about 20 m²/g to about 50 m²/g, about 20 m²/g to about 60 m²/g, about 20 m²/g to about 100 m²/g, about 20 m²/g to about 80 m²/g, about 40 m²/g to about 50 m²/g, about 40 m²/g to about 80 m²/g, about 40 m²/g to about 100 m²/g, or about 40 m²/g to about 60 m²/g. In certain embodiments of the first, second, or third aspect, the aluminium doped zinc oxide or composite may have a specific surface area of, for example, about 10, 11, 12, 15, 20, 30, 40, 45, 50, 55, 60, 65, 70, 75, 80, 90, or 100 m²/g.

In certain embodiments of the first, second, or third aspect, the zinc oxide is mesoporous. It may have a total mesopore volume of from about 0.2 cm3/g to about 0.9 cm3/g, or from about 0.25 cm3/g to about 0.9 cm3/g, about 0.30 cm3/g to about 0.9 cm3/g, about 0.4 cm3/g to about 0.9 cm3/g, about 0.5 cm3/g to about 0.9 cm3/g, about 0.4 cm3/g to about 0.8 cm3/g, about 0.4 cm3/g to about 0.7 cm3/g, or about 0.4 cm3/g to about 0.6 cm3/g. In certain embodiments of the first, second, or third aspect, the zinc oxide may have a total mesopore volume of at least about 0.2, 0.25, 0.3, 0.4, 0.45, 0.5, 0.55, 0.6, 0.65, 0.7, 0.75, 0.8, or 0.85 cm3/g. In certain embodiments, the zinc oxide may, for example, have a total mesopore volume of about 0.2, 0.25, 0.3, 0.35, 0.4, 0.45, 0.47, 0.49, 0.5, 0.51, 0.52, 0.55, 0.57, 0.6, 0.65, 0.7, 0.75, 0.8, 0.85, or 0.9 cm3/g. In certain embodiments of the first, second, or third aspect, the zinc oxide has a total mesopore volume of at least about 0.25 cm3/g. The average pore size in the zinc oxide may be from about 2 nm to about 100 nm, or it may be from about 5 nm to about 100 nm, about 10 nm to about 100 nm, about 20 nm to about 100 nm, about 2 nm to about 80 nm, about 2 nm to about 60 nm, about 20 nm to about 80 nm, about 20 nm to about 60 nm, or about 40 nm to about 60 nm. It may, for example, have an average pore size of about 2, 5, 10, 11, 12, 15, 20, 30, 40, 50, 55, 60, 65, 70, 80, 90, or 100 nm.

In certain embodiments of the first, second, or third aspect, the zinc oxide may have a specific surface area of from about 10 m²/g to about 100 m²/g, or it may be from about 20 m²/g to about 100 m²/g, about 20 m²/g to about 80 m²/g, about 40 m²/g to about 100 m²/g, or about 40 m²/g to about 80 m²/g. In certain embodiments of the first, second, or third aspect, the zinc oxide may have a specific surface area of, for example, about 10, 11, 12, 15, 20, 30, 40, 45, 50, 55, 60, 65, 70, 75, 80, 90, or 100 m²/g.

In certain embodiments of the first, second, or third aspect, the aluminium oxide may be in the form of particles having a plate-like shape. The aluminium oxide particles may have a mean aspect ratio of at least about 1.5, or at least about 2, 3, 4, 5, 7, 8, or 9. It may be from about 1.5 to about 10, or from about 1.5 to about 8, about 1.5 to about 6, about 1.5 to about 4, about 1.5 to about 2, about 2 to about 5, about 2 to about 4, or about 2 to about 10. It may be about 1.5, 1.6, 1.7, 1.8, 1.9, 1, 1.1, 1.2, 2, 3, 4, 5, 6, 7, 8, 9, or 10. The aspect ratio may be defined as the ratio of the minimum non-thickness dimension to the average thickness. The aluminium oxide particles may be non-uniform in shape, but on average may have non-thickness dimension at least 1.5 times greater than its average thickness. In certain embodiments of the first, second, or third aspect, the aluminium oxide comprises Alusion™.

The mass ratio of aluminium oxide to zinc oxide in the calcinated mixture may be from about 1:4 to about 1:24, or from about 1:4 to about 1:5, about 1:4 to about 1:10, about 1:4 to about 1:15, about 1:8 to about 1:20, about 1:10 to about 1:20, or about 1:15 to about 1:24. It may be, for example, about 1:4, 1:4.5, 1:5, 1:6; 1:7; 1:8; 1:9; 1:10; 1:12; 1:14; 1:16; 1:18; 1:20; or 1:24. In certain embodiments of the first, second, or third aspect, the mass ratio of aluminium oxide to zinc oxide in the calcinated mixture is from about 1:4 to about 1:24.

The aluminium oxide may have an average non-thickness dimension of from about 0.1 μm to about 10 μm, or it may be from about 0.1 μm to about 5 μm, about 0.5 μm to about 10 μm, about 0.5 μm to about 10 μm, or about 0.5 μm to about 5 μm. It may be, for example, about 0.1, 0.2, 0.5, 1, 1.1, 1.2, 1.5, 2, 5, or 10 μm. In certain embodiments of the first, second, or third aspect, the aluminium oxide has an average non-thickness dimension of from about 0.1 to about 50 μm.

The aluminium oxide may have an average thickness of from about 10 nm to about 3000 nm, or it may be from about 10 nm to about 1000 nm, about 10 nm to about 500 nm, about 10 nm to about 250 nm, about 20 nm to about 1000 nm, about 20 nm to about 500 nm, about 20 nm to about 250 nm, about 50 nm to about 1000 nm, about 50 nm to about 500 nm, or about 50 nm to about 250 nm. It may be, for example, about 10, 20, 50, 100, 200, 300, 400, 500, 600, 700, 800, 900, 1000, 2000, or 3000 nm. In certain embodiments of the first, second, or third aspect, the aluminium oxide has an average thickness of from about 50 to about 250 nm.

In certain embodiments of the second or third aspect, the sunscreen composition is an organic sunscreen composition. In this context, the term “organic” means that the sunscreen complies with the definition of “organic” or “organic derived” in ISO16128-1:2016, or ISO16128-2:2017; or that it complies with the definition of “organic” in the scope of ECOCERT COSMOS, or NSF/ANSI 305-2016. In one embodiment, the sunscreen does not comprise any ingredients that originate from a fossil fuel.

In certain embodiments of the second or third aspect, the sunscreen composition is a vegan sunscreen composition. In other words, in certain embodiments the sunscreen composition does not comprise any animal products, or any products that are derived from animals or animal products. In certain embodiments the sunscreen composition does not include any ingredients of animal origin.

In certain embodiments of the second or third aspect, the sunscreen composition has a Sun Protection Factor (SPF) rating of from about 20 to about 80, or from about 21 to about 80, about 22 to about 80, about 23 to about 80, about 26 to about 80, about 29 to about 80, about 32 to about 80, about 35 to about 80, about 38 to about 80, about 41 to about 80, about 44 to about 80, about 47 to about 80, about 50 to about 80, about 20 to about 78, about 20 to about 76, about 20 to about 74, about 20 to about 72, about 20 to about 70, about 20 to about 68, about 20 to about 66, about 20 to about 64, about 20 to about 62, about 20 to about 60, about 50 to about 60, about 52 to about 60, about 54 to about 60, about 56 to about 60, about 58 to about 60, about 50 to about 58, about 50 to about 56, about 50 to about 54, or about 50 to about 52. It may be greater than or equal to about 20, 21, 21, 21, 22, 23, 26, 29, 32, 35, 38, 41, 44, 47, 50, 51, 52, 53, or 54. It may be less than or equal to about 80, 78, 76, 74, 72, 70, 68, 66, 64, 55, 60, 59, 58, 57, or 56. In certain embodiments, it may be, for example, about 20, 21, 22, 23, 26, 29, 32, 35, 38, 41, 44, 47, 50, 50, 51, 52, 53, 54, 55, 56, 57, 58, 59, 60, 64, 66, 68, 70, 72, 74, 76, 78, or 80.

The sunscreen composition according to the third aspect may incorporate the sunscreen base according to the first aspect. The sunscreen base according to the first aspect may be a component of the sunscreen composition according to the third aspect.

In a fourth aspect of the invention, there is provided a method of producing a sunscreen composition, said method comprising the following steps:
contacting a calcinated mixture of zinc oxide and aluminium oxide with a carrier oil, and a dispersing and/or wetting agent to form a dispersion; and
contacting the dispersion with an aqueous phase to form the sunscreen composition.

The following options may be used in conjunction with the fourth aspect, either individually or in any combination.

In certain embodiments, the calcinated mixture, dispersing and/or wetting agent, and/or aqueous phase may be as hereinbefore described with respect to the second aspect. In certain embodiments, the aluminium oxide, and/or zinc oxide may be as hereinbefore described with respect to the second aspect.

In certain embodiments, the dispersion further comprises an emulsifier. The emulsifier may be as hereinbefore described with respect to the second aspect.

In certain embodiments, the method further comprises the following steps:
forming a blend of zinc carbonate and aluminium oxide; and
calcinating the blend at a temperature sufficient to convert the zinc carbonate to zinc oxide to thereby form the calcinated mixture.

In certain embodiments, the calcinated mixture comprises an aluminium doped zinc oxide or composite. The aluminium doped zinc oxide or composite may be as hereinbefore described with respect to the first or second aspect.

In certain embodiments, the zinc carbonate is mesoporous. It may have a total mesopore volume of from about 0.2 cm3/g to about 0.9 cm3/g, or from about 0.25 cm3/g to about 0.9 cm3/g, about 0.30 cm3/g to about 0.9 cm3/g, about 0.4 cm3/g to about 0.9 cm3/g, about 0.5 cm3/g to about 0.9 cm3/g, about 0.4 cm3/g to about 0.8 cm3/g, about 0.4 cm3/g to about 0.7 cm3/g, or about 0.4 cm3/g to about 0.6 cm3/g. In certain embodiments, the zinc carbonate may have a total mesopore volume of at least about 0.2, 0.25, 0.3, 0.4, 0.45, 0.5, 0.55, 0.6, 0.65, 0.7, 0.75, 0.8, or 0.85 cm3/g. In certain embodiments, the zinc carbonate may, for example, have a total mesopore volume of about 0.2, 0.25, 0.3, 0.35, 0.4, 0.45, 0.47, 0.49, 0.5, 0.51, 0.52, 0.55, 0.57, 0.6, 0.65, 0.7, 0.75, 0.8, 0.85, or 0.9 cm3/g. In certain embodiments, the zinc carbonate has a total mesopore volume of at least about 0.25 cm3/g. The average pore size in the zinc carbonate may be from about 2 nm to about 100 nm, or it may be from about 5 nm to about 100 nm, about 10 nm to about 100 nm, about 20 nm to about 100 nm, about 2 nm to about 80 nm, about 2 nm to about 60 nm, about 20 nm to about 80 nm, about 20 nm to about 60 nm, or about 40 nm to about 60 nm. It may, for example, have an average pore size of about 2, 5, 10, 11, 12, 15, 20, 30, 40, 50, 55, 60, 65, 70, 80, 90, or 100 nm.

In certain embodiments, the zinc carbonate may have a specific surface area of from about 10 m²/g to about 100 m²/g, or it may be from about 15 m²/g to about 50 m²/g, about 10 m²/g to about 50 m²/g, about 20 m²/g to about 50 m²/g, about 20 m²/g to about 40 m²/g, or about 20 m²/g to about 30 m²/g. In certain embodiments, the zinc carbonate may have a specific surface area of, for example, about 10, 11, 12, 15, 20, 30, 40, 45, 50, 70, 80, 90, or 100 m²/g.

In certain embodiments, the method may further comprise a step of reacting a zinc halide salt with a carbonate salt to form the zinc carbonate. The zinc halide salt may be zinc chloride. The carbonate salt may be an alkali metal salt, preferably sodium carbonate or potassium carbonate. The step of reacting the zinc halide salt with a carbonate salt may be prior to the forming a blend of zinc carbonate and aluminium oxide step in the method described herein.

In certain embodiments, the method may further comprise a step of reacting a zinc sulfate salt with a carbonate salt to form the zinc carbonate. The carbonate salt may be an alkali metal salt, preferably sodium carbonate or potassium carbonate. The step of reacting the zinc sulfate salt with a carbonate salt may be prior to the forming a blend of zinc carbonate and aluminium oxide step in the method described herein. In certain embodiments, the method further comprises a step of reacting zinc ash with sulfuric acid to form the zinc sulfate salt. The step of reacting the zinc ash with sulfuric acid may be prior to the step of reacting the zinc sulfate salt with the carbonate salt to form the zinc carbonate.

In certain embodiments, the method may further comprise a step of calcinating aluminium hydroxide in the presence of a diluent to form the aluminium oxide. The diluent may preferably be sodium chloride or sodium sulfate. The calcination may be performed at a temperature of about 700° C. or more. The step of calcinating aluminium hydroxide in the presence of a diluent may be prior to the forming a blend of zinc carbonate and aluminium oxide step in the method described herein.

In certain embodiments, the method comprises the following steps:
reacting a zinc halide salt with a carbonate salt to form zinc carbonate;
heating aluminium hydroxide in the presence of a diluent to form aluminium oxide;
forming a blend of the zinc carbonate and aluminium oxide; and
calcinating the blend at a temperature sufficient to convert the zinc carbonate to zinc oxide to thereby form the calcinated mixture.

In certain embodiments, the blend is calcinated at a sufficiently low temperature to retain mesoporosity in the zinc oxide. In certain embodiments, the zinc oxide has a total mesopore volume of at least about 0.25 cm3/g.

The calcinating may be performed in any suitable calcinating apparatus known in the art. For example, the calcinating may be performed using a kiln, such as an electric kiln.

The temperature may be in the range of about 250° C. to about 600° C., about 300° C. to about 600° C., about 350° C. to about 600° C., about 250° C. to about 575° C., about 250° C. to about 550° C., or about 250° C. to about 500° C. It may be, for example, about 250, 300, 350, 400, 450, 500, 550, 575, or 600° C. In certain embodiments, the temperature is in the range of from about 250° C. to about 575° C.

The blend may be calcinated at the temperature for a period of from about 2 hours to about 12 hours, or from about 2.5 hours to about 12 hours, about 2 hours to about 10 hours, about 2 hours to about 8 hours, about 4 hours to about 6 hours; or about 2 hours to about 6 hours. It may be, for example, about 2, 2.5, 3, 3.5, 4, 4.5, 5, 5.5, 6, 7, 8, 9, 10, 11, or 12 hours. In certain embodiments, the blend is calcinated at the temperature for a period of from about 4 hours to about 6 hours. After calcinating at the temperature, the method may further comprise a cooling step, wherein the calcinated mixture is cooled, preferably to room temperature.

The blend may be calcinated to the temperature at a heating rate of from about 5° C./hour to about 500° C./hour, or from about 10° C./hour to about 500° C./hour, about 20° C./hour to about 500° C./hour, about 50° C./hour to about 500° C./hour, about 5° C./hour to about 200° C./hour, about 10° C./hour to about 200° C./hour, about 20° C./hour to about 200° C./hour, about 50° C./hour to about 200° C./hour, or about 50° C./hour to about 150° C./hour. It may be, for example, about 10, 20, 50, 60, 70, 80, 90, 100, 110, 120, 150, 200, 300, 400, or 500° C./hour.

The mass ratio of aluminium oxide to zinc carbonate in the blend may be from about 1:5 to about 1:25, or from about 1:5 to about 1:20, about 1:5 to about 1:10, about 1:5 to about 1:9, about 1:5 to about 1:8, about 1:5 to about 1:7, or about 1:5 to about 1:6. It may be, for example, about 1:5, 1:55, 1:57, 1:6, 1:6.5, 1:7, 1:8, 1:9, 1:10, 1:12, 1:14, 1:16, 1:18, 1:20, 1:22, 1:24, or 1:25. In certain embodiments, the mass ratio of aluminium oxide to zinc carbonate in the blend is from about 1:5 to about 1:25.

In certain embodiments, the method further comprises a communition step. The communition step may comprise grinding or milling the calcinated mixture and/or the blend.

The sunscreen composition according to the second or third aspect may be produced using the method according to the fourth aspect. The method according to the fourth aspect may produce the sunscreen composition according to the second or third aspect.

In a fifth aspect of the invention, there is provided a method of protecting skin from UV irradiation, the method comprising applying the sunscreen composition according to the second or third aspect.

The following options may be used in conjunction with the fifth aspect, either individually or in any combination.

In certain embodiments, the sunscreen composition is applied to a surface, e.g. skin. In certain embodiments, the sunscreen composition is essentially transparent when applied to a surface, e.g. skin, in the form of a thin film.

Disclosed herein are the following forms:

1. A sunscreen base, comprising:
a calcinated mixture comprising aluminium oxide and zinc oxide;
a dispersing and/or wetting agent; and
a carrier oil.
   2. The sunscreen base of form 1, further comprising tropolone, or a derivative thereof, optionally hinokitiol.
   3. The sunscreen base of form 2, comprising from about 0.2 wt. % to about 5 wt. % hinokitiol.
   4. The sunscreen base of any one of forms 1 to 3, wherein the calcinated mixture comprises an aluminium doped zinc oxide or an aluminium oxide-zinc oxide composite.
   5. The sunscreen base of any one of forms 1 to 4, wherein the zinc oxide is mesoporous.
   6. The sunscreen base of form 5, wherein the zinc oxide has a total mesopore volume of at least about 0.25 cm3/g.
   7. The sunscreen base of any one of forms 1 to 6, wherein the aluminium oxide has a plate-like shape.
   8. The sunscreen base of any one of forms 1 to 7, wherein the mass ratio of aluminium oxide to zinc oxide in the calcinated mixture is from about 1:4 to about 1:24.
   9. The sunscreen base of any one of forms 1 to 8, wherein the aluminium oxide has an average non-thickness dimension of from about 0.1 to about 10 μm.
   10. The sunscreen base of any one of forms 1 to 9, comprising from about 15 wt. % to about 50 wt. % of the carrier oil.
   11. The sunscreen base of any one of forms 1 to 10, further comprising an emulsifier.
   12. The sunscreen base of form 11, comprising from about 2.5 wt. % to about 10 wt. % of the emulsifier.
   13. The sunscreen base of form 11 or 12, wherein the emulsifier comprises polyglyceryl-3 polyricinoleate.
   14. The sunscreen base of any one of forms 1 to 13, wherein the carrier oil comprises Helianthus annuus seed oil.
   15. The sunscreen base of any one of forms 1 to 14, wherein the dispersing and/or wetting agent comprises isostearic acid.
   16. The sunscreen base of any one of forms 1 to 15, comprising from about 0.2 wt. % to about 2 wt. % of the dispersing and/or wetting agent.
   17. The sunscreen base of any one of forms 1 to 16, comprising from about 10 wt. % to about 50 wt. % of the calcinated mixture.
   18. The sunscreen base of any one of forms 1 to 15, further comprising pristine zinc oxide.
   19. The sunscreen base of form 18, comprising from about 10 wt. % to about 50 wt. %, optionally from about 10 wt. % to about 25 wt. %, of the pristine zinc oxide.
   20. A sunscreen composition, comprising:
a calcinated mixture comprising aluminium oxide and zinc oxide;
an emulsifier;
a dispersing and/or wetting agent;
a carrier oil; and
an aqueous phase.
   21. The sunscreen composition of form 20, further comprising tropolone, or a derivative thereof, optionally hinokitiol.
   22. The sunscreen composition of form 21, comprising from about 0.1 wt. % to about 2.5 wt. % hinokitiol.
   23. The sunscreen composition of any one of forms 20 to 22, wherein the calcinated mixture comprises an aluminium doped zinc oxide or an aluminium oxide-zinc oxide composite.
   24. The sunscreen composition of any one of forms 20 to 23, wherein the zinc oxide is mesoporous.
   25. The sunscreen composition of form 24, wherein the zinc oxide has a total mesopore volume of at least about 0.25 cm3/g.
   26. The sunscreen composition of any one of forms 20 to 25, wherein the aluminium oxide has a plate-like shape.
   27. The sunscreen composition of any one of forms 20 to 26, wherein the mass ratio of aluminium oxide to zinc oxide in the calcinated mixture is from about 1:4 to about 1:24.
   28. The sunscreen composition of any one of forms 20 to 27, wherein the aluminium oxide has an average non-thickness dimension of from about 0.1 to about 10 μm.
   29. The sunscreen composition of any one of forms 20 to 28, comprising from about 10 wt. % to about 35 wt. % of the carrier oil.
   30. The sunscreen composition of any one of forms 20 to 29, comprising from about 20 wt. % to about 40 wt. % of the aqueous phase.
   31. The sunscreen composition of any one of forms 20 to 30, comprising from about 2 wt. % to about 5 wt. % of the emulsifier.
   32. The sunscreen composition of any one of forms 20 to 31, comprising from about 0.2 wt. % to about 1.5 wt. % of the dispersing and/or wetting agent.
   33. The sunscreen composition of any one of forms 20 to 32, comprising from about 5 wt. % to about 30 wt. % of the calcinated mixture.
   34. The sunscreen composition of any one of forms 20 to 33, further comprising pristine zinc oxide.
   35. The sunscreen composition of form 34, comprising from about 5 wt. % to about 40 wt. % of the pristine zinc oxide.
   36. The sunscreen composition of any one of forms 20 to 35, further comprising one or more additional components selected from the group consisting of humectants, salts, chelating agents, stabilizing agents, texturizing agents, emollients, film forming agents, occlusive agents, and water-resistant agents.
   37. The sunscreen composition of any one of forms 20 to 36, wherein the emulsifier comprises polyglyceryl-3 polyricinoleate.
   38. The sunscreen composition of any one of forms 20 to 37, wherein the carrier oil comprises Helianthus annuus seed oil.
   39. The sunscreen composition of any one of forms 20 to 38, wherein the dispersing and/or wetting agent comprises isostearic acid.
   40. A sunscreen composition comprising:
from about 10 wt. % to about 20 wt. % Helianthus annuus seed oil;
from about 2 wt. % to about 5 wt. % polyglyceryl-3 polyricinoleate;
from about 0.2 wt. % to about 3 wt. % isostearic acid;
from about 10 wt. % to about 20 wt. % calcinated aluminium and zinc oxide;
from about 0 wt. % to about 7 wt. % of coconut alkanes and cococaprylate/caprate;
from about 10 wt. % to about 40 wt. %, optionally from about 10 wt. % to about 25 wt. %, pristine zinc oxide;
from about 1 wt. % to about 5 wt. % jojoba oil;
from about 0.1 wt. % to about 2 wt. % polyhydroxystearic acid;
from about 0 wt. % to about 1 wt. % acrylate octylacrylamide copolymer;
from about 1 wt. % to about 5 wt. % wax;
from about 0 wt. % to about 5 wt. % soya oil;
from about 0.1 wt. % to about 1 wt. % xanthan gum;
from about 20 wt. % to about 40 wt. % water;
from about 5 wt. % to about 15 wt. % glycerin;
from about 0.1 wt. % to about 5 wt. % maltodextrin;
from about 0.1 wt. % to about 5 wt. % potassium cetyl phosphate;
from about 0.1 wt. % to about 5 wt. % sodium chloride; and
from about 0.01 wt. % to about 1 wt. % disodium EDTA.
   41. A sunscreen composition comprising the following components:

| Ingredient Name | wt. % Range |
|---|---|
| Calcinated mixture of Zinc Oxide and | 23-35 |
| Aluminium Oxide | |
| Helianthus Annuus Seed Oil | 15-25 |
| Simmondsia Chinensis (Jojoba) Seed oil | 2-6 |
| Polyglyceryl-3 Polyricinoleate | 1-5 |
| Polyhydroxy Stearic Acid | 0.5-1 |
| Wax | 1-5 |
| Isostearic Acid | 0.5-2 |
| Xanthan Gum | 0.5-2 |
| Sodium Chloride | 0.5-2 |
| Glycerin | 10-15 |
| Maltodextrin | 0.5-2 |
| Potassium Cetyl Phosphate | 0.5-2 |
| Water | Up to 100 |

42. A sunscreen composition comprising the following components:

| Ingredient Name | wt. % Range | |
|---|---|---|
| Calcinated mixture of Zinc Oxide and | 25 to | 40 |
| Aluminium Oxide | | |
| Sunflower Oil | 15 to | 25 |
| Jojoba oil | 2 to | 5 |
| PolyGlyceryl Polyricinoleate (PGPR) | 2 to | 5 |
| Polyhydroxystearic Acid | 0.5 to | 5 |
| Hinokitiol | 0.25 to | 2 |
| Wax | 0.25 to | 3 |
| Isostearic Acid | 0.5 to | 3 |
| Xanthan Gum | 0.1 to | 2 |
| Water | 20 to | 35 |
| Glycerin | 5 to | 15 |
| Maltodextrin | 0.5 to | 3 |
| Amphisol K | 0.1 to | 2 |
| Sodium Chloride | 0.1 to | 5 |
| Sodium Phytate | 0.1 to | 0.5 |

43. A method of producing a sunscreen composition, said method comprising the following steps:
contacting a calcinated mixture of zinc oxide and aluminium oxide with a carrier oil, and a dispersing and/or wetting agent to form a dispersion; and
contacting the dispersion with an aqueous phase to form the sunscreen composition.
   44. The method of form 43, wherein the dispersion further comprises an emulsifier.
   45. The method of form 43 or 44, further comprising the following steps:
forming a blend of zinc carbonate and aluminium oxide; and
calcinating the blend at a temperature sufficient to convert the zinc carbonate to zinc oxide to thereby form the calcinated mixture.
   46. A method of protecting skin from UV irradiation, the method comprising applying the sunscreen composition of any one of forms 20 to 42 to said skin.

### BRIEF DESCRIPTION OF THE DRAWINGS

FIG. 1 shows a scanning electron micrograph of aluminium oxide plate-like particles produced in Example 1b.

FIG. 2 shows: (A) reflectance spectra of: (a) calcinated mixture comprising zinc oxide and aluminium oxide; (b) physical blend of zinc oxide and aluminium oxide; (c) zinc oxide; (d) aluminium oxide; and (B) expanded section of FIG. 2(A).

FIG. 3 shows Scanning Electron Microscopy (SEM) & Energy Dispersive Spectroscopy (EDS) of a physical blend of zinc oxide and aluminium oxide:
(A) SEM image of physical blend of zinc oxide and aluminium oxide showing the positions where EDS analyses were performed (Spectra 18-26);
(B) EDS analysis performed at the position indicated as Spectrum 18 in FIG. 3(A);
(C) EDS analysis performed at the position indicated as Spectrum 19 in FIG. 3(A);
(D) EDS analysis performed at the position indicated as Spectrum 20 in FIG. 3(A);
(E) EDS analysis performed at the position indicated as Spectrum 21 in FIG. 3(A);
(F) EDS analysis performed at the position indicated as Spectrum 22 in FIG. 3(A);
(G) EDS analysis performed at the position indicated as Spectrum 23 in FIG. 3(A);
(H) EDS analysis performed at the position indicated as Spectrum 24 in FIG. 3(A);
(I) EDS analysis performed at the position indicated as Spectrum 25 in FIG. 3(A);
(J) EDS analysis performed at the position indicated as Spectrum 26 in FIG. 3(A);
(K) SEM image of physical blend of zinc oxide and aluminium oxide: scale line 100 μm; EHT 20 kV; WD 7.7 mm; Signal A BSD; Aperture Size 60 μm;
(L) SEM image of physical blend of zinc oxide and aluminium oxide: scale line 10 μm; EHT 5 kV; WD 4.8 mm; Signal A InLens; Aperture Size 30 μm;
(M) SEM image of physical blend of zinc oxide and aluminium oxide: scale line 2 μm; EHT 5 kV; WD 4.8 mm; Signal A InLens; Aperture Size 30 μm;
(N) SEM image of physical blend of zinc oxide and aluminium oxide: scale line 1 μm; EHT 5 kV; WD 4.8 mm; Signal A InLens; Aperture Size 30 μm;
(O) SEM image of physical blend of zinc oxide and aluminium oxide: scale line 1 μm; EHT 5 kV; WD 4.8 mm; Signal A SE2; Signal B InLens; Aperture Size 30 μm;
(P) SEM image of physical blend of zinc oxide and aluminium oxide: scale line 200 nm; EHT 5 kV; WD 4.8 mm; Signal A InLens; Aperture Size 30 μm;
(Q) SEM image of physical blend of zinc oxide and aluminium oxide: scale line 200 nm; EHT 5 kV; WD 4.8 mm; Signal A SE2; Signal B InLens; Aperture Size 30 μm;
(R) SEM image of physical blend of zinc oxide and aluminium oxide: scale line 200 nm; EHT 5 kV; WD 4.8 mm; Signal A SE2; Signal B InLens; Aperture Size 30 μm.

FIG. 4 shows Scanning Electron Microscopy (SEM) & Energy Dispersive Spectroscopy (EDS) of a calcinated mixture of zinc oxide and aluminium oxide:
(A) SEM image of calcinated mixture of zinc oxide and aluminium oxide showing the positions where EDS analyses were performed (Spectra 9-17);
(B) EDS analysis performed at the position indicated as Spectrum 9 in FIG. 4(A);
(C) EDS analysis performed at the position indicated as Spectrum 10 in FIG. 4(A);
(D) EDS analysis performed at the position indicated as Spectrum 11 in FIG. 4(A);
(E) EDS analysis performed at the position indicated as Spectrum 12 in FIG. 4(A);
(F) EDS analysis performed at the position indicated as Spectrum 13 in FIG. 4(A);
(G) EDS analysis performed at the position indicated as Spectrum 14 in FIG. 4(A);
(H) EDS analysis performed at the position indicated as Spectrum 15 in FIG. 4(A);
(I) EDS analysis performed at the position indicated as Spectrum 16 in FIG. 4(A);
(J) EDS analysis performed at the position indicated as Spectrum 17 in FIG. 4(A);
(K) SEM image of calcinated mixture of zinc oxide and aluminium oxide: scale line 100 μm; EHT 20 kV; WD 7.6 mm; Signal A BSD; Aperture Size 60 μm;
(L) SEM image of calcinated mixture of zinc oxide and aluminium oxide: scale line 10 μm; EHT 5 kV; WD 4.8 mm; Signal A InLens; Aperture Size 30 μm;
(M) SEM image of calcinated mixture of zinc oxide and aluminium oxide: scale line 2 μm; EHT 5 kV; WD 4.8 mm; Signal A InLens; Aperture Size 30 μm;
(N) SEM image of calcinated mixture of zinc oxide and aluminium oxide: scale line 1 μm; EHT 5 kV; WD 4.8 mm; Signal A InLens; Aperture Size 30 μm;
(O) SEM image of calcinated mixture of zinc oxide and aluminium oxide: scale line 200 nm; EHT 5 kV; WD 4.8 mm; Signal A InLens; Aperture Size 30 μm;
(P) SEM image of calcinated mixture of zinc oxide and aluminium oxide: scale line 1 μm; EHT 5 kV; WD 4.8 mm; Signal A SE2; Signal B InLens; Aperture Size 30 μm;
(Q) SEM image of calcinated mixture of zinc oxide and aluminium oxide: scale line 200 nm; EHT 5 kV; WD 4.8 mm; Signal A SE2; Signal B InLens; Aperture Size 30 μm;
(R) SEM image of calcinated mixture of zinc oxide and aluminium oxide: scale line 200 nm; EHT 5 kV; WD 4.8 mm; Signal A SE2; Signal B InLens; Aperture Size 30 μm.

FIG. 5 includes graphical representations of the SPF data from Table 3:
(A) In FIG. 5A, points represent individual formulations, with lines connecting paired formulations (i.e. formulations having identical amounts of components except for hinokitiol);
(B) FIG. 5B shows that the SPF increase at 4 weeks for formulations with hinokitiol is greater than in the formulations without hinokitiol; and
(C) FIG. 5C shows that the SPF increase at 12 weeks is greater in formulations with hinokitiol than in formulations without hinokitiol.

### DEFINITIONS

In describing and claiming the present invention, the following terminology will be used in accordance with the definitions set out below. It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Unless the context clearly requires otherwise, throughout the description and the claims, the terms “comprise”, “comprising”, and the like are to be construed in an inclusive sense as opposed to an exclusive or exhaustive sense; that is to say, in the sense of “including, but not limited to”. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition, process or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising”, it should be readily understood that (unless otherwise stated) the description should be interpreted to also describe such an invention using the terms “consisting essentially of” or “consisting of.” In other words, with respect to the terms “comprising”, “consisting of”, and “consisting essentially of”, where one of these three terms is used herein, the presently disclosed and claimed subject matter may include the use of either of the other two terms. Thus, in some embodiments not otherwise explicitly recited, any instance of “comprising” may be replaced by “consisting of” or, alternatively, by “consisting essentially of”.

Other than in the operating examples, or where otherwise indicated, all numbers expressing quantities of ingredients or reaction conditions used herein are to be understood as modified in all instances by the term “about”. The examples are not intended to limit the scope of the invention. In what follows, or where otherwise indicated, “%” will mean “weight %”, “ratio” will mean “weight ratio” and “parts” will mean “weight parts”.

The terms “predominantly”, “predominant”, and “substantially” as used herein shall mean comprising more than 50% by weight, unless otherwise indicated.

As used herein, with reference to numbers in a range of numerals, the terms “about,” “approximately” and “substantially” are understood to refer to the range of −10% to +10% of the referenced number, preferably −5% to +5% of the referenced number, more preferably −1% to +1% of the referenced number, most preferably −0.1% to +0.1% of the referenced number. Moreover, with reference to numerical ranges, these terms should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, from 8 to 10, and so forth.

The terms “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the term “mesoporous” refers to pores ranging in size from about 2 nm to about 100 nm. The pores are categorized as “open pores” that connect through and open onto a surface of the material or as “closed pores” that are sealed from fluid ingress from the surface of the material. The distribution of pore sizes and total pore volume of the material may be measured using gas adsorption and pycnometry or other techniques which are known to those of skill in the art.

As used herein, the term “calcinated mixture” with respect to the “calcinated mixture comprising zinc oxide and aluminium oxide”, means a mixture comprising zinc oxide and aluminium oxide that has been produced by calcinating a mixture of zinc carbonate and aluminium oxide at a temperature sufficient to convert the zinc carbonate to zinc oxide; that is, the calcinating occurs after the zinc and aluminium complexes are mixed. In certain embodiments, the temperature may be from about 250° C. to about 575° C. In certain embodiments, the “calcinated mixture” may comprise aluminium doped zinc oxide or an aluminium oxide-zinc oxide composite.

As used herein, the term “carbonate” with respect to “zinc carbonate”, includes any carbonate-containing zinc salts. For example, “zinc carbonate” includes ZnCO₃, Zn₅(OH)₆(CO₃)₂, and Zn(HCO₃)₂.

As used herein, the term “at. %” refers to atomic percentage.

The term “alkyl” refers to a straight-chain or branched alkyl substituent containing from, for example, 1 to about 36 carbon atoms. Examples of suitable alkyl groups include, but are not limited to, methyl, ethyl, propyl, isopropyl, n-butyl, sec-butyl, isobutyl, tert-butyl, pentyl, isoamyl, 2-methylbutyl, 3-methylbutyl, hexyl, heptyl, 2-methylpentyl, 3-methylpentyl, 4-methylpentyl, 2-ethylbutyl, 3-ethylbutyl, octyl, nonyl, decyl, undecyl, dodecyl and the like. The number of carbons referred to relates to the carbon backbone and carbon branching but does not include carbon atoms belonging to any substituents, for example the carbon atoms of an alkoxy substituent branching off the main carbon chain.

The term “alkenyl” refers to a straight-chain or branched alkenyl substituent containing from, for example, 2 to about 36 carbon atoms. Examples of suitable alkenyl groups include, but are not limited to, ethenyl, propenyl, isopropenyl, butenyl, butadienyl, pentenyl, pentadienyl, hexenyl, hexadienyl, heptenyl, octenyl, nonenyl, decenyl, undecenyl, dodecenyl and the like. The number of carbons referred to relates to the carbon backbone and carbon branching but does not include carbon atoms belonging to any substituents, for example the carbon atoms of an alkoxy substituent branching off the main carbon chain.

The term “alkynyl” refers to a straight-chain or branched alkynyl substituent containing from, for example, 2 to about 36 carbon atoms. Examples of suitable alkynyl groups include, but are not limited to, ethynyl, propynyl, butynyl, butadienyl, pentynyl, hexynyl, heptynyl, octynyl, nonynyl, decynyl, undecynyl, dodecynyl and the like. The number of carbons referred to relates to the carbon backbone and carbon branching but does not include carbon atoms belonging to any substituents, for example the carbon atoms of an alkoxy substituent branching off the main carbon chain.

The term “cycloalkyl” refers to a saturated non-aromatic cyclic hydrocarbon. The cycloalkyl ring may include a specified number of carbon atoms. For example, a 3 to 8 membered cycloalkyl group includes 3, 4, 5, 6, 7 or 8 carbon atoms. The cycloalkyl group may be monocyclic, bicyclic or tricyclic. When more than one ring is present the rings are fused together (for example, a bicyclic ring is fused if two atoms are common to both rings). Non-limiting examples may include cyclopropyl, cyclobutyl, cyclopentyl, cyclohexyl and the like.

The term “cycloalkenyl” or “cycloalkene” refers to a cyclic hydrocarbon having at least one double bond, which is not aromatic. The cycloalkenyl ring may include a specified number of carbon atoms. The cycloalkenyl group may be monocyclic, bicyclic or tricyclic. When more than one ring is present the rings are fused together (for example, a bicyclic ring is fused if two atoms are common to both rings). For example, a 5 membered cycloalkenyl group includes 5 carbon atoms. Non-limiting examples may include cyclopentenyl and cyclopenta-1,3-dienyl.

The term “cycloalkynyl” or “cycloalkyne” refers to a cyclic hydrocarbon having at least one triple bond, which is not aromatic. The cycloalkynyl ring may include a specified number of carbon atoms. The cycloalkynyl group may be monocyclic, bicyclic or tricyclic. When more than one ring is present the rings are fused together (for example, a bicyclic ring is fused if two atoms are common to both rings). For example, a 5 membered cycloalkynyl group includes 5 carbon atoms. Non-limiting examples may include cyclopentynyl.

The term “aryl” refers to an aromatic carbocyclic substituent, as commonly understood in the art. It is understood that the term aryl applies to cyclic substituents in which at least one ring is planar and comprises 4n+2 π electrons, according to Hückel's Rule. Aryl groups may be monocyclic, bicyclic or tricyclic. Examples of aryl groups include, but are not limited to, phenyl, naphthyl and 1,2,3,4-tetrahydronaphthyl. An aryl group may be monocyclic, bicyclic or tricyclic, provided that at least one ring is aromatic. When more than one ring is present the rings are fused together (for example, a bicyclic ring is fused if two atoms are common to both rings).

The term “heteroalkyl” refers to a straight-chain or branched alkyl substituent in which one or more carbon atoms have been replaced by heteroatoms independently selected from N, S and O. It may contain from, for example, 1 to about 36 carbon atoms. For example, between 1 and 4 carbon atoms may be replaced by heteroatoms independently selected from N, S and O. Examples of suitable heteroalkyl groups include, but are not limited to, methoxy, ethoxy, propyloxy, isopropyloxy, and the like. The number of carbons referred to relates to the carbon backbone and carbon branching but does not include carbon atoms belonging to any substituents, for example the carbon atoms of an alkoxy substituent branching off the main carbon chain.

The term “heteroalkenyl” refers to a straight-chain or branched alkenyl substituent in which one or more carbon atoms have been replaced by heteroatoms independently selected from N, S and O. It may contain from, for example, 2 to about 36 carbon atoms. For example, between 1 and 4 carbon atoms may be replaced by heteroatoms independently selected from N, S and O. The number of carbons referred to relates to the carbon backbone and carbon branching but does not include carbon atoms belonging to any substituents, for example the carbon atoms of an alkoxy substituent branching off the main carbon chain.

The term “heteroalkynyl” refers to a straight-chain or branched alkynyl substituent in which one or more carbon atoms have been replaced by heteroatoms independently selected from N, S and O. It may contain from, for example, 2 to about 36 carbon atoms. For example, between 1 and 4 carbon atoms may be replaced by heteroatoms independently selected from N, S and O. The number of carbons referred to relates to the carbon backbone and carbon branching but does not include carbon atoms belonging to any substituents, for example the carbon atoms of an alkoxy substituent branching off the main carbon chain.

The term “heterocyclic” or “heterocyclyl” as used herein, refers to a cycloalkyl or cycloalkenyl group in which one or more carbon atoms have been replaced by heteroatoms independently selected from N, S and O. For example, between 1 and 4 carbon atoms in each ring may be replaced by heteroatoms independently selected from N, S and O. The heterocyclyl group may be monocyclic, bicyclic or tricyclic in which at least one ring includes a heteroatom. When more than one ring is present the rings may be fused together (for example, a bicyclic ring is fused if two atoms are common to both rings). Each of the rings of a heterocyclyl group may include, for example, between 5 and 7 atoms. Examples of heterocyclyl groups include tetrahydrofuranyl, tetrahydrothiophenyl, pyrrolidinyl, pyrrolinyl, dithiolyl, 1,3-dioxanyl, dioxinyl, piperidinyl, piperazinyl, morpholinyl, thiomorpholinyl, pyranyl, 1,4-dithiane, and decahydroisoquinoline. In one embodiment, heterocyclyl may be optionally substituted by ═O.

The term “heteroaryl”, as used herein, refers to a monocyclic, bicyclic or tricyclic ring of up to 7 atoms in each ring, wherein at least one ring is aromatic and said at least one ring contains from 1 to 4 heteroatoms selected from the group consisting of O, N and S. When more than one ring is present the rings are fused together (for example, a bicyclic ring is fused if two atoms are common to both rings). Consideration must be provided to tautomers of heteroatom containing ring systems containing carbonyl groups, for example, when determining if a ring is a heterocyclyl or heteroaryl ring. Heteroaryl includes, but is not limited to, 5-membered heteroaryls having one hetero atom (e.g., thiophenes, pyrroles, furans); 5 membered heteroaryls having two heteroatoms in 1,2 or 1,3 positions (e.g., oxazoles, pyrazoles, imidazoles, thiazoles, purines); 5-membered heteroaryls having three heteroatoms (e.g., triazoles, thiadiazoles); 5-membered heteroaryls having four heteroatoms (e.g., tetrazoles); 6-membered heteroaryls with one heteroatom (e.g., pyridine, quinoline, isoquinoline); 6-membered heteroaryls with two heteroatoms (e.g., pyridazines, cinnolines, phthalazines, pyrazines, pyrimidines, quinazolines, quinoxalinone, quinazolinone); 6-membered heteroaryls with three heteroatoms (e.g., 1,3,5-triazine); and 6-membered heteroaryls with four heteroatoms. Examples of heteroaryl include thiophene, benzothiophene, benzofuran, benzimidazole, benzoxazole, benzothiazole, benzisothiazole, furan, pyrrole, imidazole, pyrazole, pyridine, pyrazine, pyrimidine, pyridazine, indole, isoindole, 1H-indazole, purine, quinoline, isoquinoline, phthalazine, naphthyridine, quinoxaline, cinnoline, carbazole, phenanthridine, acridine, phenazine, thiazole, isothiazole, phenothiazine, oxazole, isooxazole, furazane, and phenoxazine. Further exemplary heteroaryl groups may include, for example, indoline or 2,3-dihydrobenzofuran. In one embodiment, heteroaryl may be optionally substituted by ═O.

Whenever a range of the number of atoms in a structure is indicated (e.g., a C₁-C₁₂, C₁-C₆ alkyl, etc.), it is specifically contemplated that any sub-range or individual number of carbon atoms falling within the indicated range also can be used. Thus, for instance, the recitation of a range of 1-12 carbon atoms (e.g., C₁-C₁₂), 1-6 carbon atoms (e.g., C₁-C₆) as used with respect to any chemical group (e.g., alkyl, etc.) referenced herein encompasses and specifically describes 1, 2, 3, 4, 5, 6, 7, 8, 9, 10, 11, and/or 12 carbon atoms, as appropriate, as well as any sub-range thereof (e.g., 1-2 carbon atoms, 1-3 carbon atoms, 1-4 carbon atoms, 1-5 carbon atoms, 1-6 carbon atoms, 1-7 carbon atoms, 1-8 carbon atoms, 1-9 carbon atoms, 1-10 carbon atoms, 1-11 carbon atoms, 1-12 carbon atoms, 2-3 carbon atoms, 2-4 carbon atoms, 2-5 carbon atoms, 2-6 carbon atoms, 2-7 carbon atoms, 2-8 carbon atoms, 2-9 carbon atoms, 2-10 carbon atoms, 2-11 carbon atoms, 2-12 carbon atoms, 3-4 carbon atoms, 3-5 carbon atoms, 3-6 carbon atoms, 3-7 carbon atoms, 3-8 carbon atoms, 3-9 carbon atoms, 3-10 carbon atoms, 3-11 carbon atoms, 3-12 carbon atoms, 4-5 carbon atoms, 4-6 carbon atoms, 4-7 carbon atoms, 4-8 carbon atoms, 4-9 carbon atoms, 4-10 carbon atoms, 4-11 carbon atoms, and/or 4-12 carbon atoms, etc., as appropriate).

As used herein, “halo” refers to a halogen atom, especially F, C₁ or Br; more especially F or C₁; most especially F.

As used herein, the term “optionally substituted” means that any number of hydrogen atoms on the optionally substituted group are replaced with another moiety. Unless defined otherwise, said moiety is independently selected from the group consisting of C₁-C₁₂ alkyl (or C₁-C₆ alkyl), C₂-C₆ alkenyl, C₂-C₆ alkynyl, aryl, C₁-C₆ heteroalkyl, C₂-C₆ heteroalkenyl, C₂-C₆ heteroalkynyl, heteroaryl, R¹²—CONH—R¹³, R¹²—NHCO—R¹³, R¹²—CSNH—R¹³, R¹²—NHCS—R¹³, R¹²—CO—R¹³, ═O, ═S, cyano, CF₃, and halogen;
wherein:
   R¹² is selected from the group consisting of C₁-C₆ alkyl, C₂-C₆ alkenyl, C₂-C₆ alkynyl, aryl, C₁-C₆ heteroalkyl, C₂-C₆ heteroalkenyl, C₂-C₆ heteroalkynyl, heteroaryl, and a bond; and
   R¹³ is selected from the group consisting of C₁-C₆ alkyl, C₂-C₆ alkenyl, C₂-C₆ alkynyl, aryl, C₁-C₆ heteroalkyl, C₂-C₆ heteroalkenyl, C₂-C₆ heteroalkynyl, heteroaryl, and hydrogen.

Preferred features, embodiments and variations of the invention may be discerned from the following Examples which provides sufficient information for those skilled in the art to perform the invention. The following Examples are not to be regarded as limiting the scope of the preceding Summary of the Invention in any way.

### EXAMPLES

### Example 1: Formation of Aluminium Oxide

The aluminium oxide used herein can be sourced commercially. Alternatively, it may be synthesised. Example procedures for synthesising aluminium oxide are described below.

### Example 1a: Al(OH)₃ in Na₂SO₄ Diluent, Stirred During Heat Treatment

180 g aluminium hydroxide as a precursor compound and 820 g sodium sulphate as a diluent were milled for 1 hour at 400 rpm in a 7 litre attrition mill using 25 kg of 6.35 mm diameter stainless steel balls to form nano-sized particles of aluminium hydroxide. 900 g of the resulting powder was added to a 4 litre alumina crucible containing 2.27 kg of pre-molten sodium sulphate at 1100° C. The mixture was mechanically stirred at 60 rpm by two alumina stirring rods during addition of the milled powder and for a further hour whilst the mixture was held at 1100° C.

X-ray diffraction and electron microscopy studies confirmed that the resulting material consisted of platelets of alpha alumina 0.5-3 microns in diameter with thickness 50-100 nm. The plate-like alumina particles were substantially discrete.

### Example 1b: Al(OH)₃ in NaCl Diluent, with Mineralizer, Solid State Heat Treatment Leading to 0.5-5 Micron Plate-Like Particles

1.04 g aluminium hydroxide as a precursor was milled with 6.88 g NaCl as a diluent and 0.08 g cryolite (Na₃AlF₆) as a mineralizer for 3 hours in a Spex mixer mill using 80 g of 12.7 mm diameter stainless steel balls to form nano-sized particles of aluminium hydroxide. Cryolite is known to be soluble in sodium chloride, forming a eutectic diluent mineralizer system, with eutectic temperature 730° C.

A sample of the resulting powder was heat treated at 500° C. for 30 minutes and washed in deionised water, in order to examine the particle size prior to transformation to alpha phase. X-ray diffraction measurements confirmed that the resulting material was gamma alumina. Electron microscopy studies revealed that the particles were equiaxed nano particles approximately 5 nm in size. The remaining powder was heat treated for 2 hours below the eutectic temperature, at 720° C.

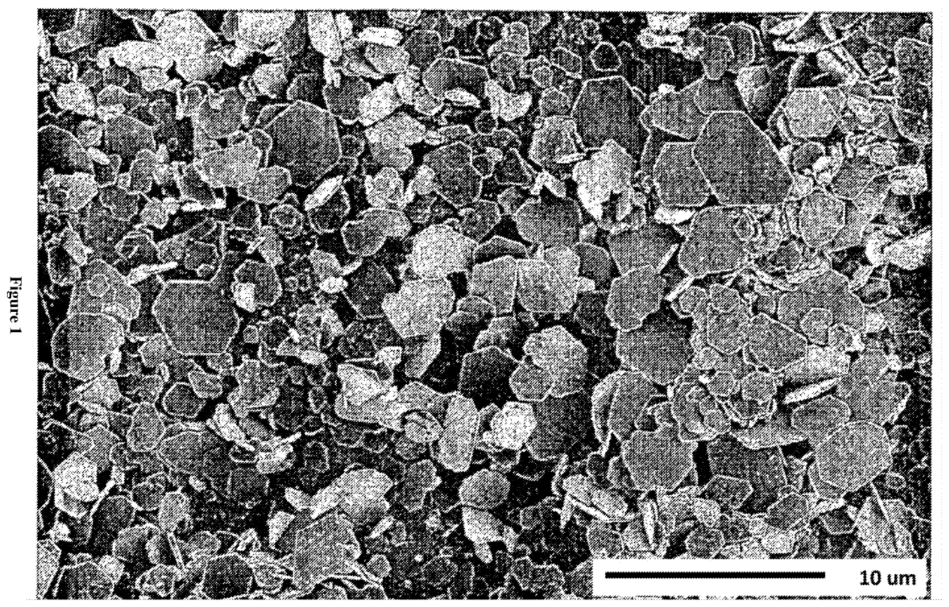

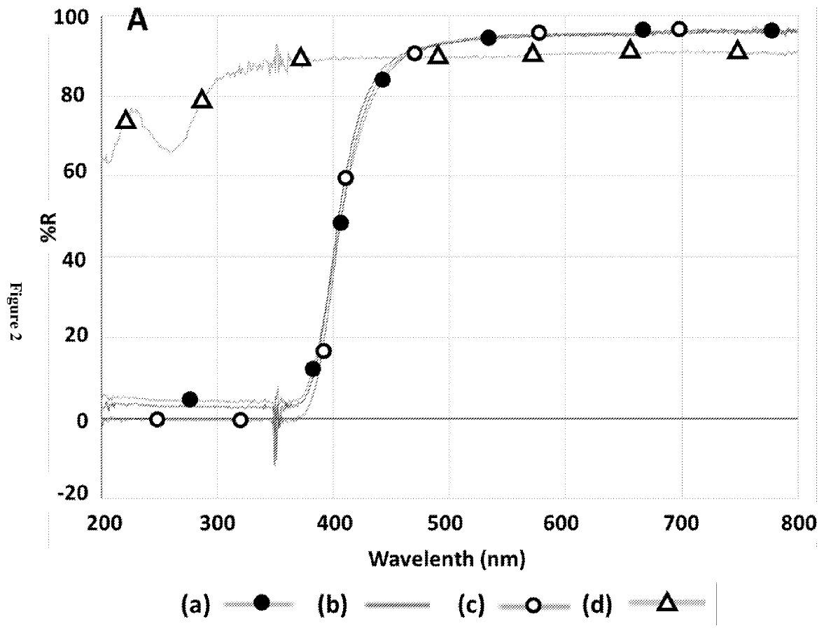

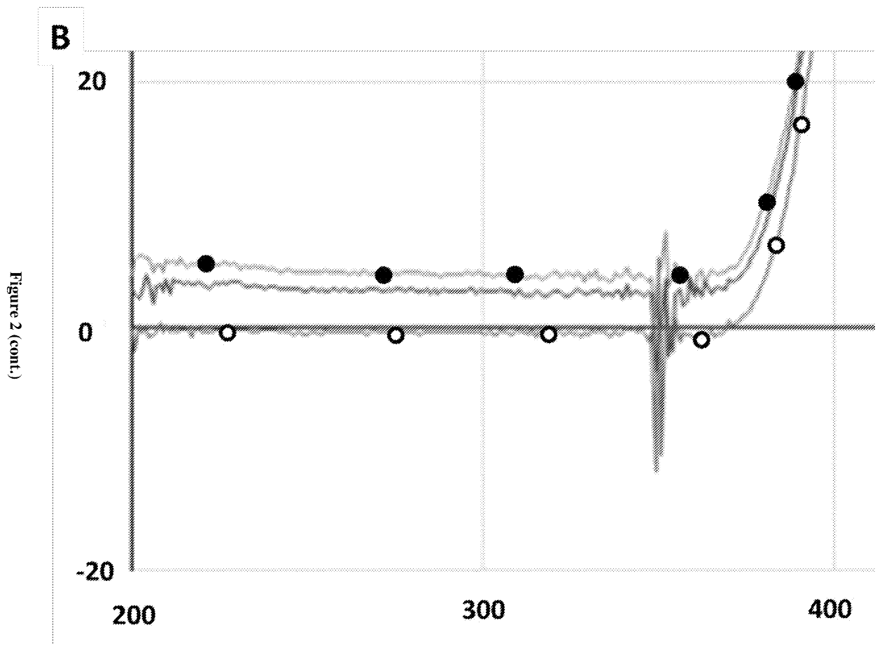

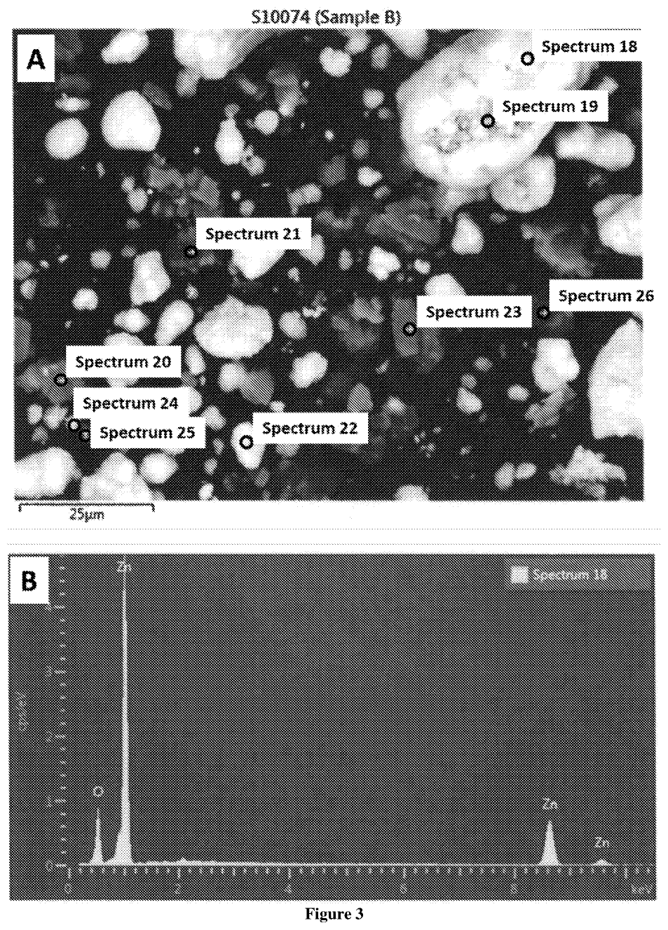

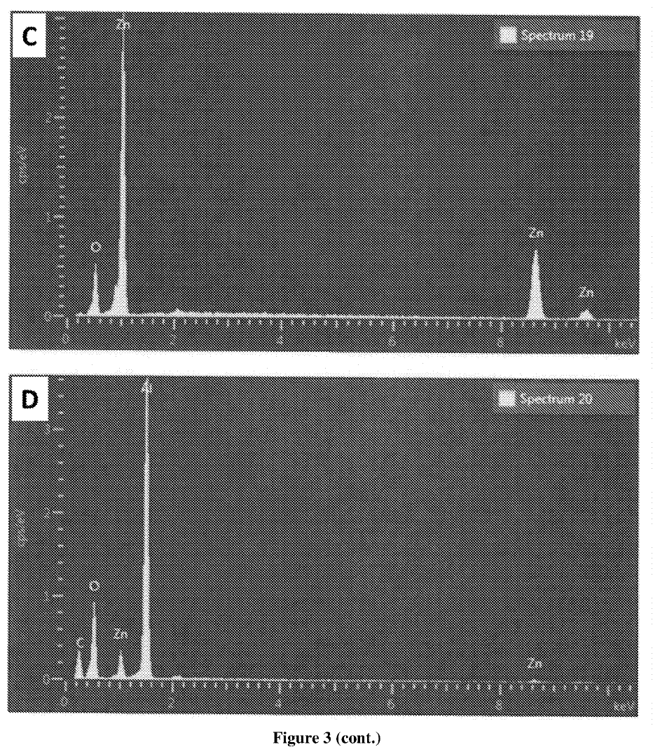

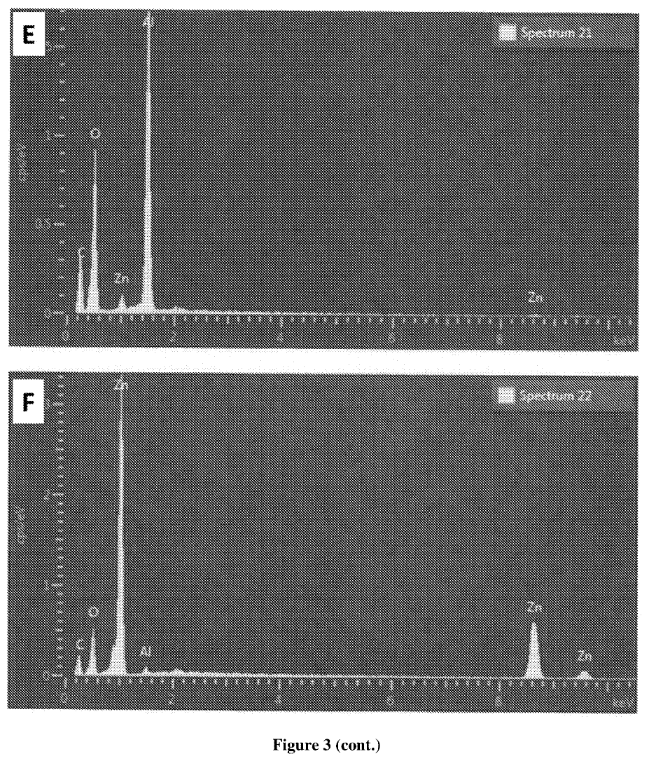

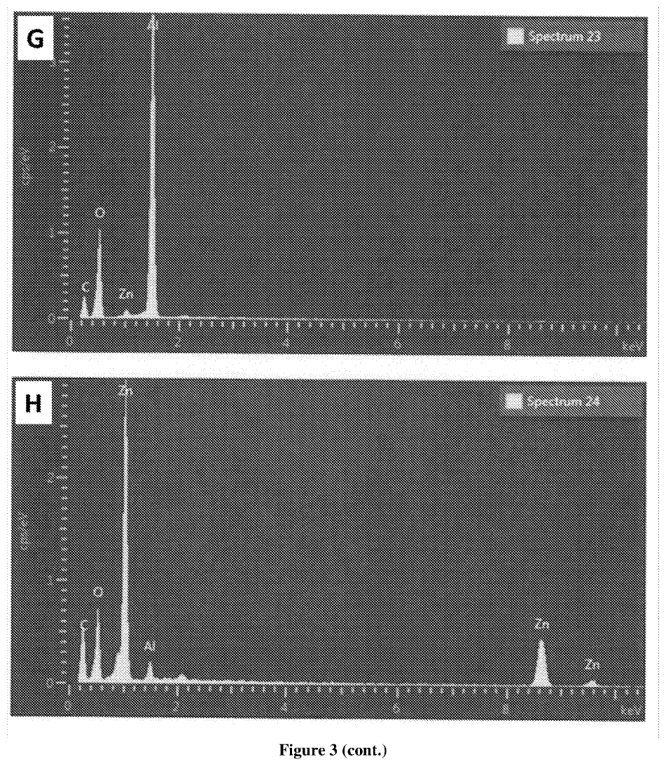

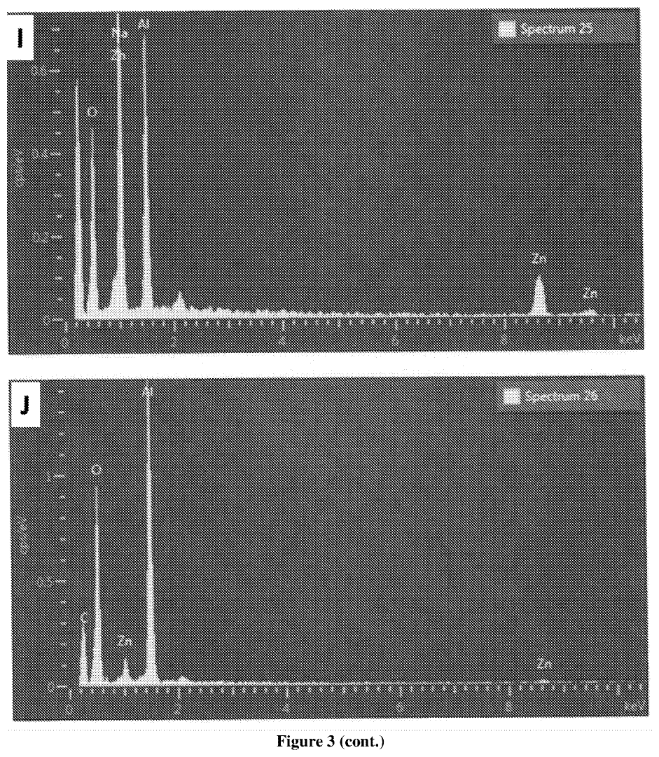

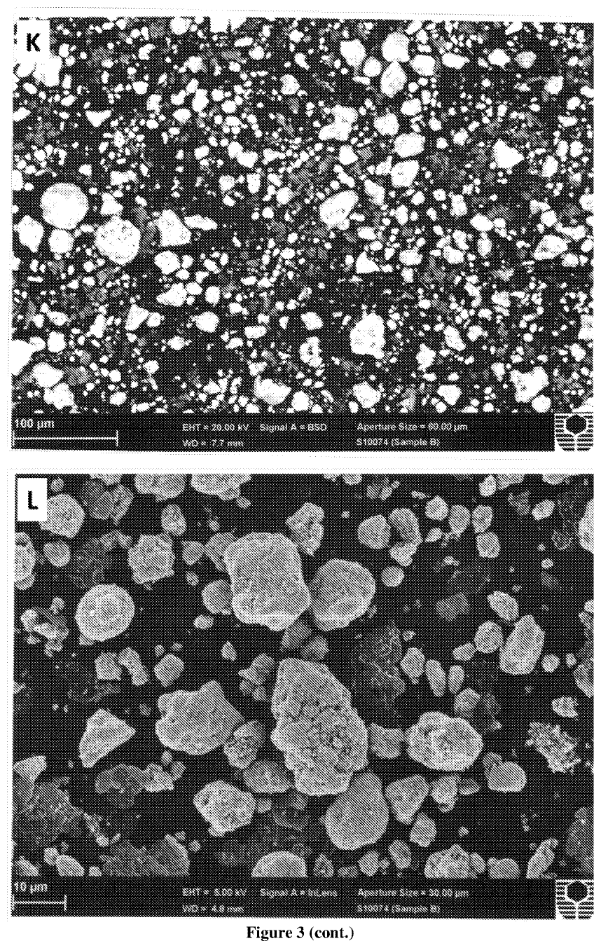

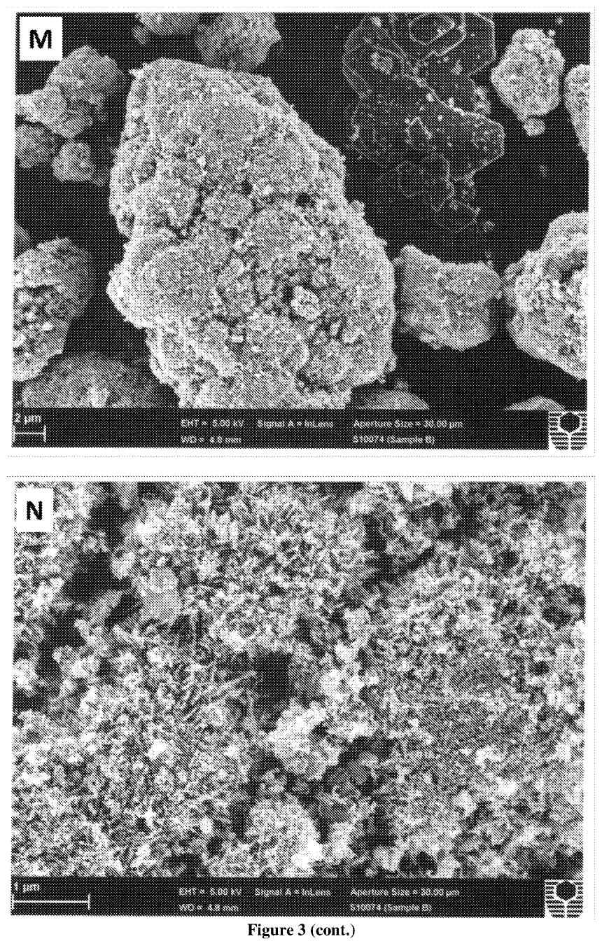

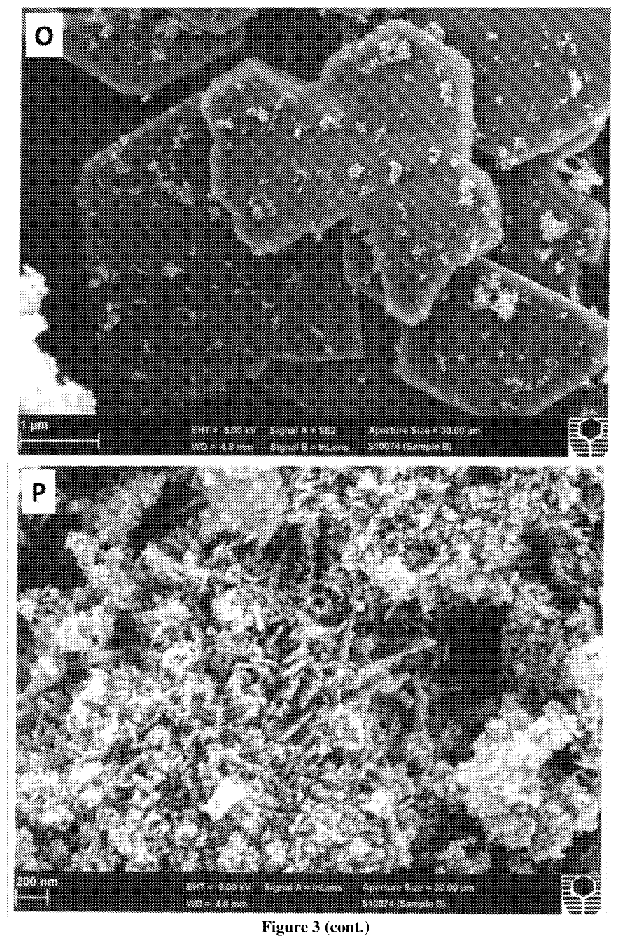

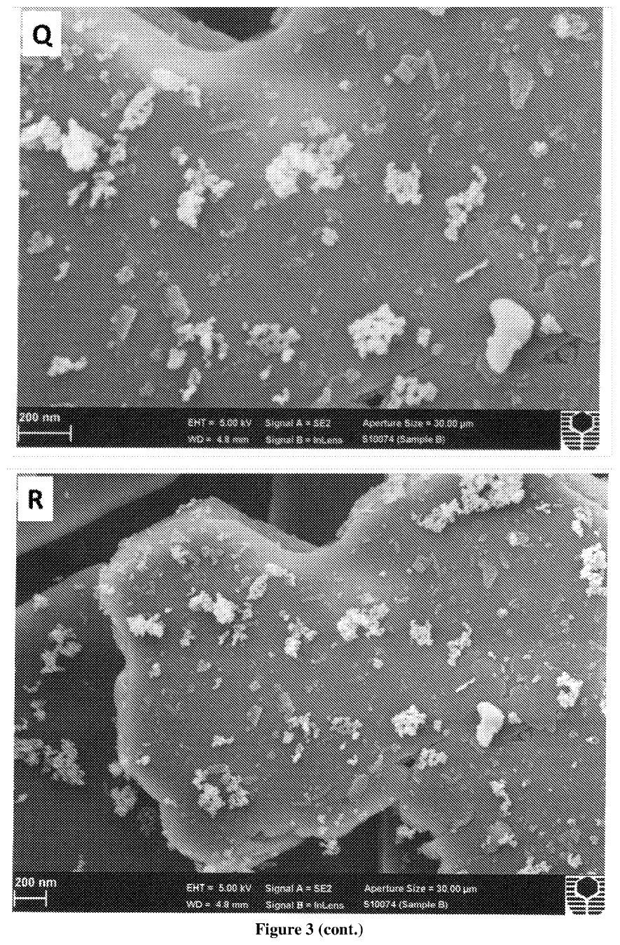

X-ray diffraction and electron microscopy studies confirmed that the resulting material consisted of platelets of alpha alumina 0.5-5 microns in diameter with thickness 50-100 nm. The particles were essentially individual platelets with low levels of agglomeration. FIG. 1 shows a scanning electron micrograph of the material produced using Example 1b.

### Example 1c: Al(OH)₃ in NaCl Diluent, with Mineralizer, Solid State Heat Treatment Below Liquidus Temperature Leading to 0.1-9 Micron Plate-Like Particles

500 g aluminium hydroxide as a precursor compound was milled with 4450 g sodium chloride as a diluent and 50 g cryolite (Na₃AlF₆) as a mineralizer for 90 minutes in a 33 litre attrition mill at 270 rpm, using 100 kg of 6.35 mm diameter stainless steel balls to form nano-sized particles of aluminium hydroxide. The liquidus temperature for diluent-mineralizer system composition is 795-800° C. The resulting powder was heat treated for 2 hours at 780° C.

X-ray diffraction and electron microscopy studies confirmed that the resulting material consisted of plate-like particles of alpha alumina 0.1-9 microns in diameter with thickness 50-150 nm. The substantially discrete particles were essentially individual platelets with low levels of aggregation.

### Example 1d: Partially Dehydrated Al(OH)₃ in NaCl Diluent with Mineralizer, Heat Treatment Below Liquidus Temperature Leading to 1-30 Micron Plate-Like Particles

650 g aluminium hydroxide as a precursor compound which had been dried to 23% mass loss at 230° C. was milled with 4300 g sodium chloride as a diluent and 50 g cryolite as a mineralizer for 90 minutes at 270 rpm in a 33 litre attrition mill using 100 kg of 6.35 mm diameter stainless steel balls to form an intermediate compound comprising nano-sized particles of aluminium hydroxide.

X-ray diffraction measurements showed that the starting hydroxide material was predominantly boehmite (AlOOH), with a small fraction of gibbsite (AI (OH) 3) remaining. The resulting powder was heat treated for 2 hours at 780° C.

X-ray diffraction and electron microscopy studies confirmed that the resulting material consisted of platelets of alpha alumina 1-30 microns in diameter with thickness 50-200 nm. The particles were essentially individual platelets with low levels of aggregation.

### Example 2: Preparation of Mesoporous Zinc Carbonate Precursor

Zinc carbonate precursor powder was synthesized by reacting aqueous solutions of zinc chloride and sodium carbonate in the molar ratio of 1ZnCl₂:3Na₂CO₃ at room temperature. The individual solutions consisted of 1230 g of zinc chloride dissolved in 4 L of deionized (DI) water and 960 g of sodium carbonate dissolved in 10 L of DI water. The zinc chloride solution was added under vigorous stirring to the carbonate solution resulting in a white precipitate. The precipitate was washed using deionized water to less than 100 ppm and dried at 120° C.

The crystal structure of the resulting powder was characterized by x-ray diffraction which showed the hydrozincite phase as the only phase present. Scanning electron microscope (SEM) examination of the powder showed that it consisted of mesoporous aggregates of primary crystallites. The specific surface area of the powder measured using gas adsorption (BET method, Micromeritics Tristar) was 62.4 m²/g.

The distribution of open pores was measured using gas adsorption techniques (Micromeritics Tristar) according to the Barrett-Joyner-Helenda method (described in Techniques de l'Ingenieur [Techniques of the Engineer] and entitled “Texture des solides poreux ou divises” [Texture of porous or divided solids], p. 3645-1 to 3645-13). The pore size measurements showed a distribution of pore sizes between 2 nm and 100 nm (mesopores) with the average pore size equal to 27.3 nm. The total open mesopore volume was 0.476 cm3/g.

### Example 3: Preparation of Calcinated Mixture of Zinc Oxide and Aluminium Oxide

Aluminium oxide (Alusion™) and the mesoporous zinc carbonate precursor were mixed in a mass ratio of 0.04-0.17 aluminium oxide: 0.83-0.96 zinc carbonate. The mixture was heat treated at a temperature of from about 385° C. to about 500° C. in an electric kiln. The samples were subject to slow heating with a furnace ramp rate of form about 50° C./hr to about 150° C./hr and held for from about 3 hours to about 8 hours at the set temperature to convert the zinc carbonate to zinc oxide, followed by cooling to room temperature.

### Example 4: Comparative Preparation of Calcinated Zinc Oxide without Aluminium Oxide

Zinc oxide powder was prepared from the hydrozincite powder of Example 2 by heat treating at a temperature of from about 385° C. to about 500° C. in an electric kiln. The samples were subject to slow heating with a furnace ramp rate of from about 50° C./hr to about 150° C./hr and held for from about 3 hours to about 8 hours at the set temperature, followed by cooling to room temperature. The resulting powder had an off-white colour. X-ray diffraction showed that ZnO (wurtzite phase) was the only crystalline phase present after calcining.

### Example 5: Comparison of Physical Blend of Zinc Oxide and Aluminium Oxide with Calcinated Mixture of Zinc Oxide and Aluminium Oxide

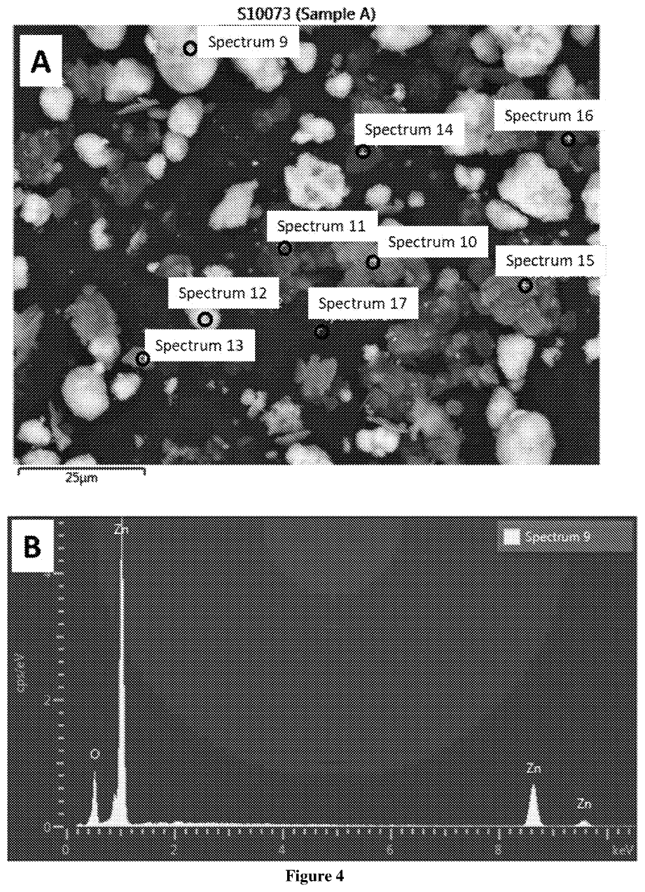

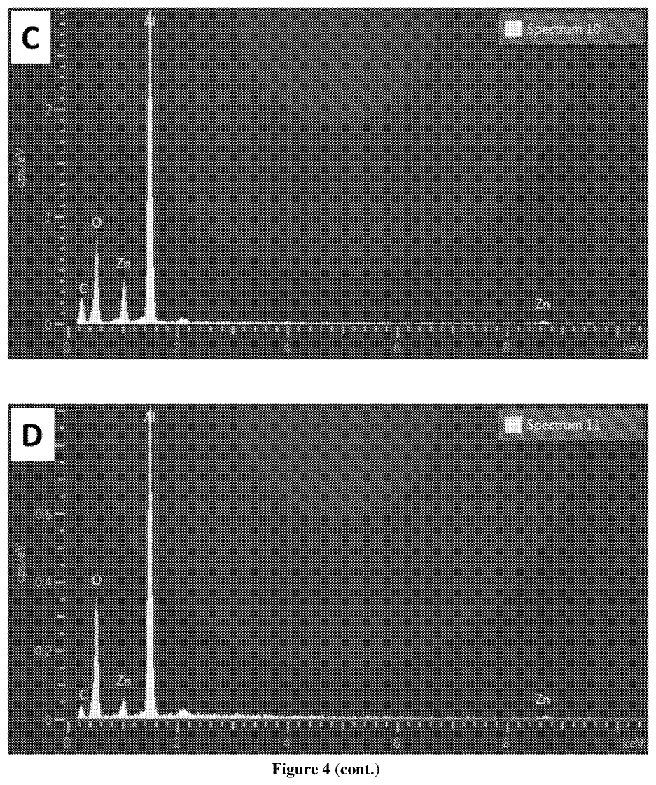

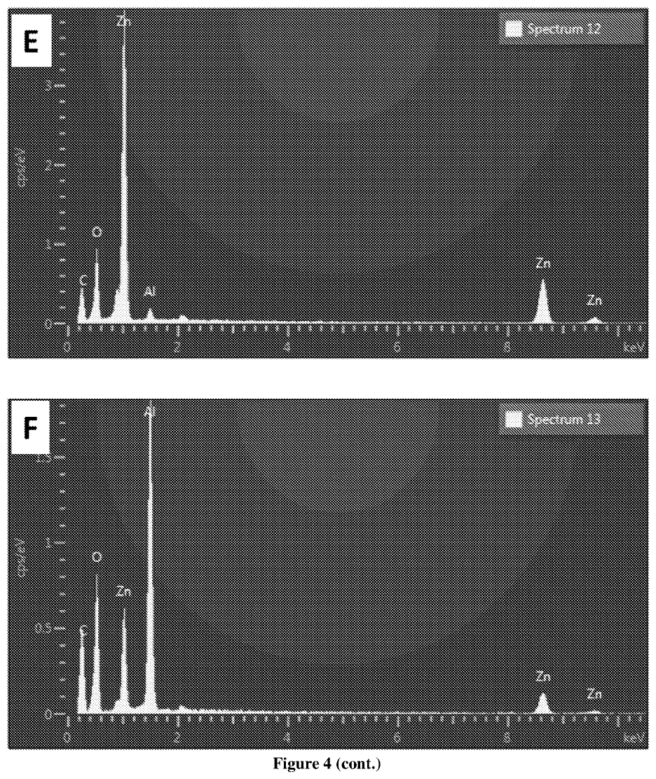

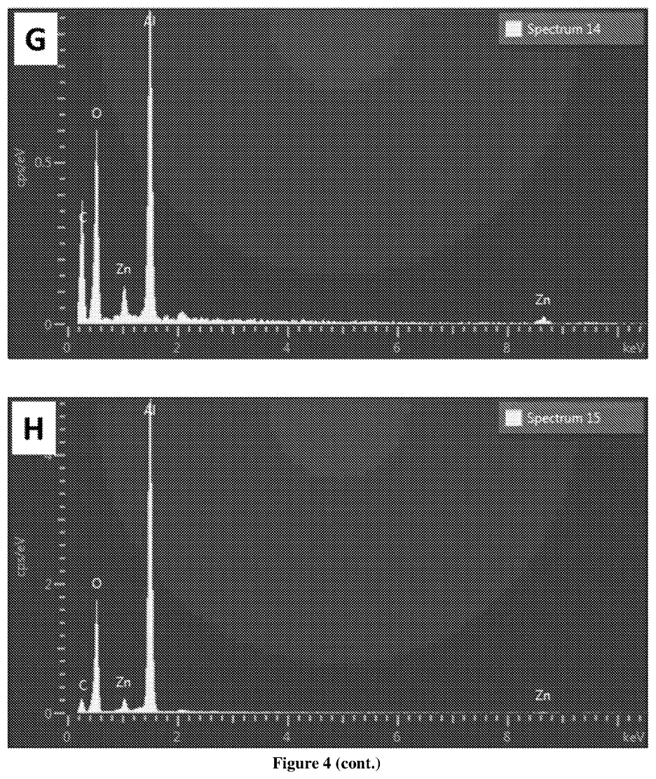

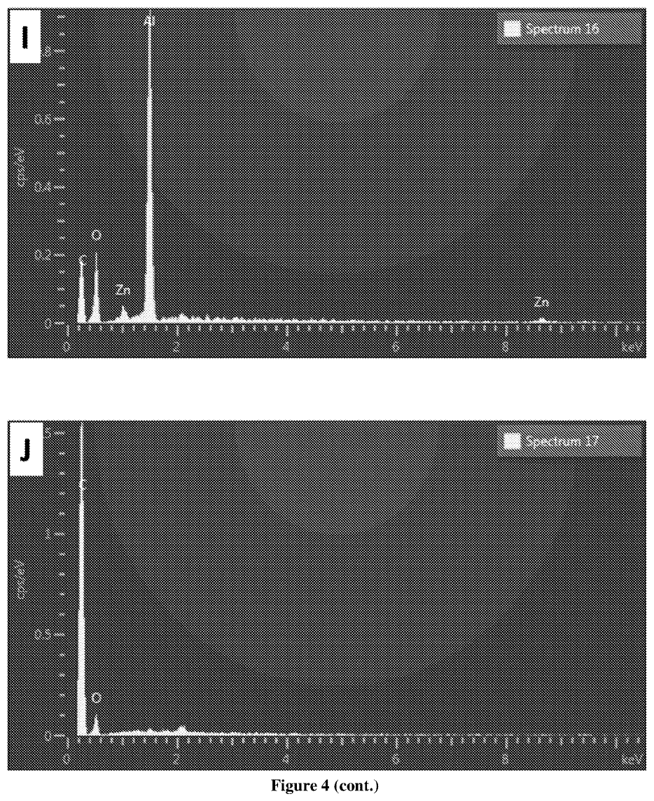

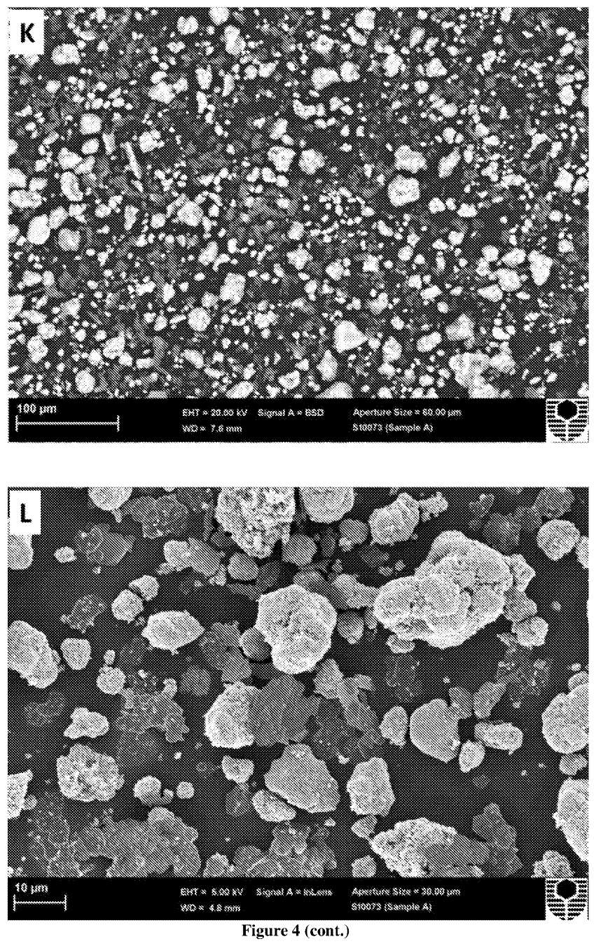

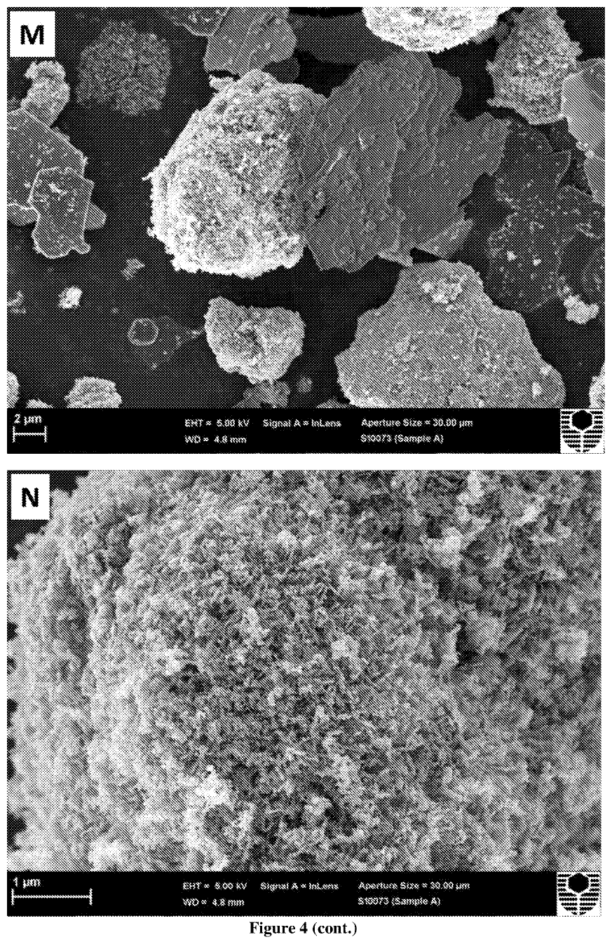

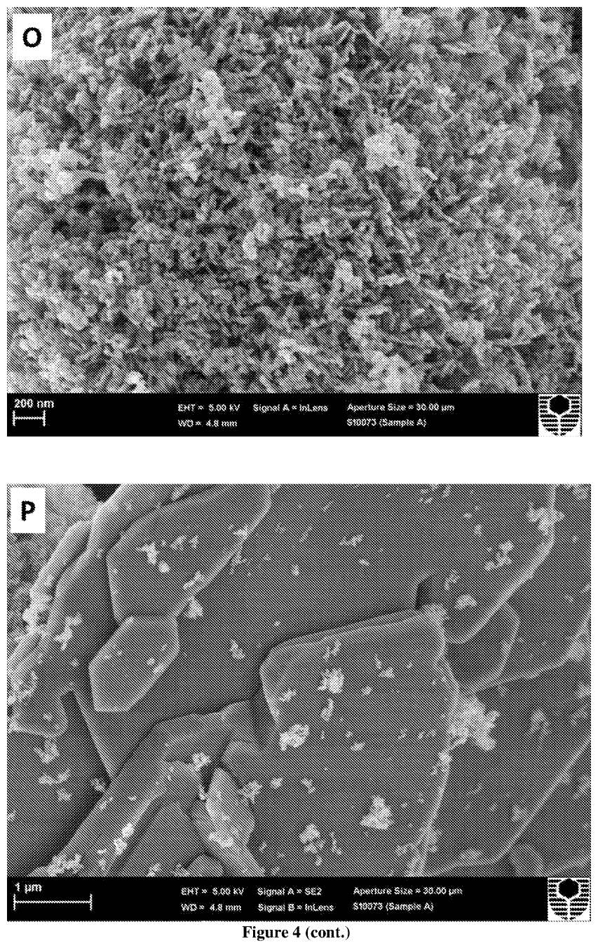

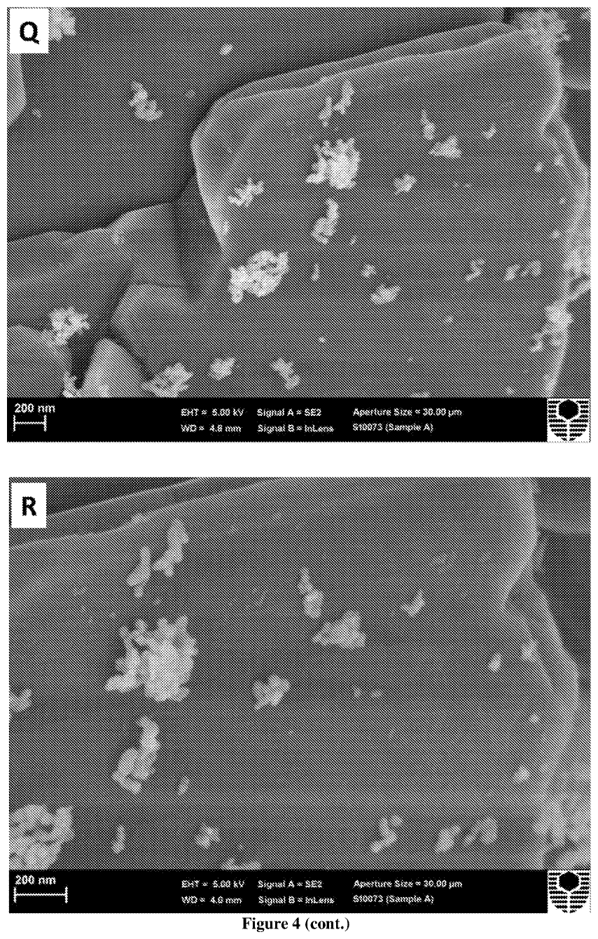

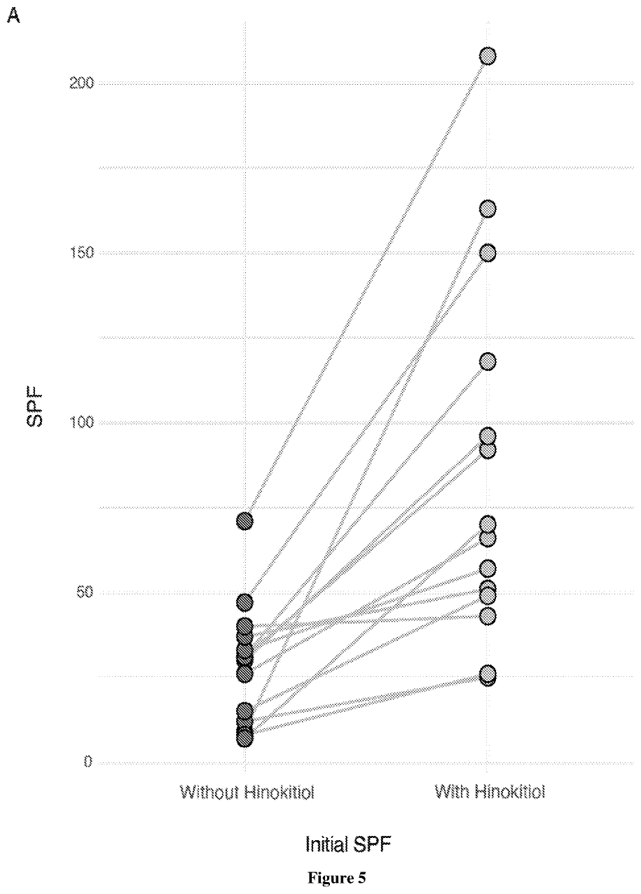

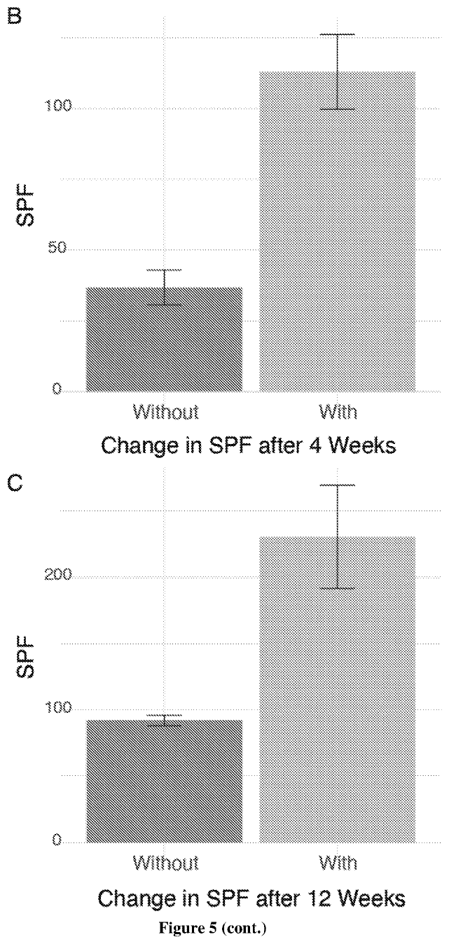

UV-visible reflectance spectroscopy shows that a calcinated mixture of zinc oxide and aluminium oxide has a higher reflectance in the UV region as compared with a physical blend of zinc oxide and aluminum oxide having the same stoichiometric ratio of zinc oxide to aluminium oxide (FIG. 2).

SEM and EDS analyses of a physical blend of zinc oxide and aluminium oxide are shown in FIG. 3. FIG. 4 shows SEM and EDS analyses of a calcinated mixture of zinc oxide and aluminium oxide.

### Example 6: Preparation of Formulations Containing Calcinated Mixture of Zinc Oxide and Aluminium Oxide

The calcinated mixture of zinc oxide and aluminium oxide was incorporated in a variety of water-in-oil emulsion-based sunscreen formulations, by mixing the calcinated mixture with the other formulation components in a heated mixture, typically at around 40-80° C.

A variety of different sunscreen formulations were prepared, including formulations with equivalent amounts (by mass) of:
(1) zinc oxide only (made according to the method outlined above at Example 4);
(2) a physical blend of zinc oxide (made according to the method outlined above at Example 4) and aluminium oxide (made according to the method outlined above at Example 1); or
(3) the inventive calcinated mixture of zinc oxide and aluminium oxide (made according to the method outlined above at Example 3).

The components and amounts for the formulations comprising zinc oxide and aluminium oxide (i.e. (2) and (3)) are set out in table 1 below:

TABLE 1
Components and amounts for the formulations
comprising zinc oxide and aluminium oxide

| Ingredient Name | wt. % Range |
|---|---|
| Zinc Oxide | 20.0-40.0 |
| Aluminium Oxide | 3.0-10.0 |
| Helianthus Annuus Seed Oil | 15.0-25.0 |
| Simmondsia Chinensis (Jojoba) Seed oil | 2.0-6.0 |
| Polyglyceryl-3 Polyricinoleate | 1.0-5.0 |
| Polyhydroxy Stearic Acid | 0.5-2.0 |
| Wax | 1.0-5.0 |
| Isostearic Acid | 0.5-3.0 |
| Xanthan Gum | 0.5-2.0 |
| Sodium Chloride | 0.5-2.0 |
| Glycerin | 5.0-15.0 |
| Maltodextrin | 0.5-3.0 |
| Potassium Cetyl Phosphate | 0.5-3 |
| Water | Up to 100 |

These formulations were tested for their SPF (sun proof factor) rating to determine the effect of the calcinating process on the SPF rating of the resultant formulation. SPF ratings for the formulations are shown in table 2 below.

Table 2: SPF results for sunscreen formulations containing: (1) zinc oxide only; (2) a physical blend of zinc oxide and aluminium oxide; or (3) the inventive calcinated mixture of zinc oxide and aluminium oxide. Formulations grouped in the same cell have equivalent amounts (by mass) of zinc oxide (in the case of (1)), or total zinc oxide and aluminium oxide (in the case of (2) or (3)), but are otherwise identical.

| | SPF rating | | |
|---|---|---|---|
| | | (2) | (3) |
| | | Physical | Calcinated |
| | (1) | blend | mixture |
| Formulation | Zinc | ZnO + | ZnO + |
| No. | oxide only | Al₂O₃ | Al₂O₃ |
| 1 | Not determined | 16 | 30 |
| 2 | 15.3 | 27 | 35 |
| 3 | 26.6 | 31 | 54 |

The SPF results for the formulations tested indicate a significant increase in SPF rating for sunscreen formulations containing the calcinated mixture of zinc oxide and aluminium oxide as compared with the physical blend of zinc oxide and aluminium oxide, and zinc oxide only formulations.

Without being bound by theory, the inventor of the present application postulate that by calcinating (heating) the zinc carbonate (to form zinc oxide) in the presence of aluminium oxide, that some of the aluminium atoms may be incorporated into, or dope the zinc oxide, resulting in a new material with improved UV absorption, UV-visible light scattering, and/or UV-visible light reflecting properties.

### Example 7: Preparation of Formulations with and without Hinokitiol

A number of sunscreen compositions were prepared, and their SPF rating was tested immediately upon preparation, four weeks after preparation and 12 weeks after preparation. The sunscreen compositions were stored at ambient temperature. The SPF results are summarised in Table 3 below:

TABLE 3
SPF results for formulations with and without hinokitiol

| | | Sample | | | | SPF after 4 | | SPF after 12 | |
|---|---|---|---|---|---|---|---|---|---|
| | | Number | | Initial SPF | | weeks | | weeks | |
| Oil | Wax | Without | With | Without | With | Without | With | Without | With |
| Coco | Wax no. 1 | 60 | 70 | 9 | 163 | | 202 | | 303 |
| Coco | Wax no. 1 | 61 | 71 | 71 | 208 | 58 | 153 | 105 | 313 |
| Coco | Wax no. 1 | 74 | 72 | 12 | 25 | | 43 | | 56 |
| Coco | Wax no. 1 | 90 | 92 | 31 | 92 | 44 | 120 | 83 | 274 |
| Coco | Wax no. 1 | 91 | 93 | 30 | 96 | 41 | 149 | 97 | 275 |
| EP | Wax no. 1 | 98 | 100 | 47 | 150 | 63 | 148 | 86 | 237 |
| EP | Wax no. 1 | 99 | 101 | 31 | 118 | 35 | 158 | 89 | 327 |
| EP | Wax no. 1 | 102 | 104 | 8 | 26 | | 43 | | 60 |
| Coco | Wax no. 1 | 134 | 159 | 26 | 66 | 44 | 127 | | |
| Coco | Wax no. 2 | 140 | 175 | 33 | 57 | 16 | 56 | | |
| Coco | Wax no. 2 | 144 | 178 | 37 | 51 | 7 | 56 | | |
| Coco | Wax no. 1 | 146 | 179 | 40 | 43 | 23 | 82 | | |
| EP | Wax no. 3 | 243 | 249 | 15 | 49 | | 114 | | |
| Sunflower | Wax no. 3 | 244 | 250 | 7 | 70 | | 131 | | |

In the above table, “coco” means coco-caprylate/caprate, “EP” means ethyl palmitate, “Sunflower” means sunflower oil. “Without” means no hinokitiol was present. “With” means that the composition contained 0.5% hinokitiol.

FIG. 5A shows a graphical representation of the data of the Table 3. It is clear from the figure that the initial SPF was increased in the formulations including hinokitiol. FIG. 5B shows that the SPF increase at 4 weeks for formulations with hinokitiol was greater than in corresponding formulations without hinokitiol. FIG. 5C shows that the SPF increase at 12 weeks was greater in formulations with hinokitiol than in corresponding formulations without hinokitiol.

Although the invention has been described with reference to specific examples, it will be appreciated by those skilled in the art that the invention may be embodied in many other forms. In particular, features of any one of the various described examples may be provided in any combination in any of the other described examples. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

1. A sunscreen base, comprising:
a calcinated mixture comprising aluminium oxide and zinc oxide;
a dispersing and/or wetting agent; and
a carrier oil.

2. The sunscreen base of claim 1, further comprising tropolone, or a derivative thereof, optionally hinokitiol optionally comprising from about 0.2 wt. % to about 5 wt. % hinokitiol.

3. (canceled)

4. The sunscreen base of claim 1, comprising from about 10 wt. % to about 50 wt. % of the calcinated mixture.

5. A sunscreen composition, comprising:
a calcinated mixture comprising aluminium oxide and zinc oxide;
an emulsifier;
a dispersing and/or wetting agent;
a carrier oil; and
an aqueous phase.

6. The sunscreen composition of claim 5, further comprising tropolone, or a derivative thereof, optionally hinokitiol optionally comprising from about 0.1 wt. % to about 2.5 wt. % hinokitiol.

7. (canceled)

8. The sunscreen composition of claim 5, comprising from about 5 wt. % to about 30 wt. % of the calcinated mixture.

9. The sunscreen composition of claim 5, further comprising pristine zinc oxide optionally comprising from about 5 wt. % to about 40 wt. %, optionally or from about 10 wt. % to about 25 wt. 9% of the pristine zinc oxide.

10. (canceled)

11. The sunscreen composition of claim 5, further comprising one or more additional components selected from the group consisting of humectants, salts, chelating agents, stabilizing agents, texturizing agents, emollients, film forming agents, occlusive agents, and water-resistant agents.

12. The sunscreen composition of claim 5, wherein the emulsifier comprises polyglyceryl-3 polyricinoleate.

13. The sunscreen composition of claim 5, wherein the dispersing and/or wetting agent comprises isostearic acid.

14. The sunscreen composition of claim 5, wherein the carrier oil comprises Helianthus annuus seed oil.

15. The sunscreen composition of claim 5, comprising the following components:

| Ingredient Name | wt. % Range | |
|---|---|---|
| Calcinated mixture of Zinc Oxide and | 25 to | 40 |
| Aluminium Oxide | | |
| Sunflower Oil | 15 to | 25 |
| Jojoba oil | 2 to | 5 |
| PolyGlyceryl Polyricinoleate (PGPR) | 2 to | 5 |
| Polyhydroxystearic Acid | 0.5 to | 5 |
| Hinokitiol | 0.25 to | 2 |
| Wax | 0.25 to | 3 |
| Isostearic Acid | 0.5 to | 3 |
| Xanthan Gum | 0.1 to | 2 |
| Water | 20 to | 35 |
| Glycerin | 5 to | 15 |
| Maltodextrin | 0.5 to | 3 |
| Amphisol K | 0.1 to | 2 |
| Sodium Chloride | 0.1 to | 5 |
| Sodium Phytate | 0.1 to | 0.5 |

16. The sunscreen composition of claim 5, wherein the calcinated mixture comprises an aluminium doped zinc oxide or an aluminium oxide-zinc oxide composite.

17. The sunscreen composition of claim 5, wherein the zinc oxide is mesoporous optionally wherein the zinc oxide has a total mesopore volume of at least about 0.25 cm3/g.

18. (canceled)

19. The sunscreen composition of claim 5, wherein the aluminium oxide has a plate-like shape.

20. The sunscreen composition of claim 5, wherein the mass ratio of aluminium oxide to zinc oxide in the calcinated mixture is from about 1:4 to about 1:24.

21. The sunscreen composition of claim 5, wherein the aluminium oxide has an average non-thickness dimension of from about 0.1 to about 10 μm.

22. (canceled)

23. A method of protecting skin from UV irradiation, the method comprising applying the sunscreen composition of claim 5 to said skin.

24. The sunscreen base of claim 1, wherein the calcinated mixture comprises an aluminium doped zinc oxide or an aluminium oxide-zinc oxide composite.

25. The sunscreen base of claim 1, wherein the zinc oxide is mesoporous, optionally wherein the zinc oxide has a total mesopore volume of at least about 0.25 cm3/g.
